(19) United States
(12) Patent Application Publication
(10) Pub. No.: US 2025/0122456 A1
(43) Pub. Date: Apr. 17, 2025

(54) MICRO-PHYSIOLOGICAL PLATFORM FOR IMMUNE RESPONSE MODELING

(71) Applicant: Javelin Biotech, Inc., Woburn, MA (US)

(72) Inventor: Murat Cirit, Cambridge, MA (US)

(21) Appl. No.: 18/896,578

(22) Filed: Sep. 25, 2024

Related U.S. Application Data
(60) Provisional application No. 63/585,150, filed on Sep. 25, 2023.

(51) Int. Cl. C12M 3/06; C12M 1/00; C12M 1/34

(52) U.S. Cl. CPC C12M 23/16; C12M 23/34; C12M 23/40; C12M 29/06; C12M 41/46

(57) ABSTRACT

A modular multi-tissue chip (MTC) microphysiological system (MPS) platform has integrated sensors and real-time data analysis software for immune engineering and CBRN studies that can be deployed by expert labs for rapid testing.

### CLAIM OF PRIORITY

This application claims priority under 35 U.S.C. § 119(e) to U.S. Patent Application Ser. No. 63/585,150, filed on Sep. 25, 2023, the entire contents of which are hereby incorporated by reference.

### TECHNICAL FIELD

This disclosure generally relates to microfluidic devices. More specifically, this disclosure relates to drug absorption and bioavailability modeling platforms.

### SUMMARY

This disclosure describes microphysiological platforms for testing, by biomimetic in vitro simulation, drug absorption in humans. The microphysiological platforms model a plurality of human tissues and enable measurements of drug absorption and bioavailability in those tissues. Various factors affecting drug absorption are modeled from the testing and various outcomes are measured, including kinetic rates.

Microphysiological systems (MPSs) can enable preparation to respond to pandemics and other chemical, biological, radiological, and nuclear (CBRN) emergencies by satisfying a need for robust systems for in vitro assessment, particularly for immune responses and continuous monitoring of tissue behavior. MPSs that replicate components of human tissues both structurally and functionally facilitate rapid testing of candidate countermeasures, identifying biomarkers or mechanisms that lead to a better understanding of injury and disease that supports emergency preparedness and rapid response capabilities against a broad set of known and unknown CBRN threats.

In an aspect, a multiple tissue chamber (MTC) system includes a first tissue chamber comprising at least a first tissue; a second tissue chamber comprising at least a second tissue different from the first tissue; an oxygenation chamber for oxygenation of fluid media configured to flow in a fluid loop through the first tissue chamber and the second tissue chamber, wherein the fluid media comprises immune cells; a pump configured for causing recirculation of the fluid media including the immune cells through the first tissue chamber and the second tissue chamber; and one or more sensors configured to measure an effect of the immune cells at the first tissue, the second tissue, or both the first tissue and the second tissue.

In some implementations, the system includes one or more sensors for trans-epithelial electrical resistance (TEER) monitoring.

In some implementations, the system includes one or more oxygen sensors configured to detect liver-specific adverse effects of mitochondrial toxicity.

In some implementations, the system includes glucose or lactate sensors configured to provide data for performance of immune assessments including T-cell activation for evaluation of adverse outcomes of CBRN threats.

In some implementations, the first tissue comprises human bronchial epithelial cells (NHBEC) on top of hydrogel-embedded human lung fibroblasts

In some implementations, the second tissue comprises CD45+ tissue-resident immune cells from spleen red and white pulp.

In some implementations, the system includes a third tissue chamber comprising a third tissue, the third tissue comprising primary small-intestinal epithelial cells expanded as organoids and seeded as monolayers with CD45+ tissue-resident immune cells from an intestinal lamina propria.

In some implementations, the first tissue chamber or the second tissue chamber or both comprise a milli-fluidic format enabling a repeated media sampling from basal recirculation and apical compartments to evaluate exposure-response relationships and-omics analysis to investigate secreted biomarkers.

In some implementations, the first tissue comprises lung tissue, the second tissue comprises liver tissue, and wherein a third tissue chamber comprises spleen tissue.

In some implementations, the first tissue comprises gut tissue, the second tissue comprises liver tissue, and wherein a third tissue chamber comprises spleen tissue.

In some implementations, the system includes a controller for receiving data from the one or more sensors to determine an immune response from the first tissue or the second tissue.

In some implementations, the controller is configured to perform quantification of on-chip pharmacokinetic (PK) parameters including hepatic metabolism, uptake & disposition, tubular secretion & reabsorption, and muscle disposition.

In some implementations, the controller is further configured to scale the quantified on-chip pharmacokinetic (PK) parameters.

In an aspect, a multiple tissue chamber (MTC) system includes a first modular micro-physiological system (MPS) system including a tissue chamber comprising at least a first tissue, the first MPS being removable from the MTC; a second MPS including a second tissue chamber comprising at least a second tissue, the second MPS being removable from the MTC, the second tissue being different from the first tissue; an oxygenation chamber for oxygenation of fluid media configured to recirculate in a fluid loop through the first tissue chamber and the second tissue chamber, a pump configured for causing recirculation of the fluid media through the first tissue chamber and the second tissue chamber; and one or more sensors configured to measure a parameter of the first tissue, the second tissue, or both the first tissue and the second tissue.

In some implementations, the system includes a controller configured to host one or more instances of the MTC system and control recirculation of the fluid media or measurement by the one or more sensors of each MTC instance.

In some implementations, the one or more sensors are configured to send data to a client device in real-time or near real-time for analysis concurrent with the recirculation of the fluid media.

In some implementations, the one or more sensors comprise electrochemical aptamer-based (E-AB) sensors 322. Electrochemical aptamer-based (E-AB) biosensors for real-time biomarker monitoring.

In some implementations, the one or more sensors a sensor array for multianalyte monitoring in real-time.

In some implementations, the first tissue comprises brain tissue, the second tissue comprises liver tissue, and wherein a third tissue chamber comprises lung tissue.

In some implementations, the first tissue comprises gut tissue, the second tissue comprises liver tissue, and wherein a third tissue chamber comprises lung tissue.

The details of one or more embodiments of these systems and methods are set forth in the accompanying drawings and the description to be presented. Other features, objects, and advantages of these systems and methods are apparent from the description and drawings, and from the claims.

### BRIEF DESCRIPTION OF THE FIGURES

FIG. 1A is a diagram showing a top view of a multi-tissue chip (MTC) including a multi-MPS microfluidic device.

FIG. 1B is a diagram showing a top view of an example of the MTC of FIG. 1A including example tissues for measuring immune responses from multiple tissues.

FIG. 2 is a diagram showing an example process for measuring immune responses.

FIG. 3A is a diagram showing an example system including the MTC of FIGS. 1A-1B.

FIG. 3B shows examples of sensors for being used with the system of FIG. 3A.

FIG. 4 shows example data representing a chronic liver inflammatory response on a multi-MPS microfluidic device.

FIG. 5 shows example data representing a chronic liver inflammatory response on an MTC.

FIG. 6A shows a perspective view of an example of an MTC.

FIG. 6B is a diagram showing a top view of an example of the MTC of FIG. 6A.

FIG. 7 is a diagram of a system including an example of an MTC.

FIG. 8 shows examples of models for testing immune responses in tissues.

FIGS. 9A-9C show examples of user interfaces.

FIG. 10 shows an example of testing an immune response using an MTC.

FIG. 11 shows data representing a pharmacokinetic CoU evaluation with small molecule drugs.

FIG. 12 shows a donor-matched model output of a gut-liver axis.

FIG. 13 is a block diagram of an example computer system.

FIG. 14 is a diagram illustrating an example computer system configured to execute a machine learning model.

### DETAILED DESCRIPTION

Human-based complex in vitro models offer the opportunity for a fundamental change in an ability to rapidly assess CBRN threats and effectively develop of medical countermeasures. Many current CBRN applications of multi-tissue chips (MTCs) can be limited to a particular testbed or experimental setup. The MTCs are generally not available for use in other experimentation or assays for testing CBRN responses in human tissues. A lack of MTC technology that is suitable for CBRN study requirements can prevent advancement of human-centric methodologies for CBRN medical countermeasure development.

The systems and methods described herein include a modular MTC platform (also called an MTC) including integrated sensors. The MTCs described herein include multi-MPS microfluidic devices (also called MPS systems or multi-MPS devices) that support human tissues and emulate responses of these tissues to CBRN threats. The MTCs described herein are configured for real-time data analysis. The analysis is configurable by a control system and user interface. The control system and user interface enable the MTC to be configured however needed for a particular emulation of human tissue response to CBRN threats. The MTCs described herein can be configured with modular components. The particular arrangement of the MPSs and sensor components of the MTC can be specified to the control system through the user interface. For example, the sample points of the fluid media, sensor types and locations, types of tissues, MPS geometries, an MPS type and their order in a fluid loop, and other configuration data are specified to the control system through the user interface for configuring a study or experiment. The MPSs can be standardized and selected from a library that is known to the control system and selectable through the user interface. A user can configure the study by arranging the MPSs on the MTC platform, specifying the desired control of circulation of the fluid media through the MPSs, and perform a custom study.

The term real-time (or similar terms as understood by one of ordinary skill in the art) means that an action and a response are temporally proximate such that an individual perceives the action and the response occurring substantially simultaneously. For example, the time difference for a response to display (or for an initiation of a display) of data following the individual's action to access the data can be less than 1 millisecond (ms), less than 1 second (s), or less than 10 s. While the requested data need not be displayed (or initiated for display) instantaneously, it is displayed (or initiated for display) without any intentional delay, accounting for processing limitations of a described computing system and time required to, for example, gather, accurately measure, analyze, process, store, or transmit the data. In another example, the MTC can measure the state of the tissues of the MPSs or a state of the fluid media, such as oxygen concentration or flow rate, and report these data to the user within a threshold time approximating a substantially simultaneous report to the measurement of the data.

The software for immune engineering and CBRN studies can be deployed by expert labs for rapid testing. In an example, an MTC platform enables integration of three MPSs and allows continuous monitoring of tissue responses by a library of integrated sensors. The integrated sensors are described in further detail below and can include transepithelial electrical resistance (TEER) sensors for barrier integrity, oxygen sensors for liver MPS health, image sensors for blood analysis, and glucose/lactate sensors for T-cell activation, among other sensors. A control system is configured to control media-based sampling for secreted biomarkers. A data processing system can process data measured by the sensors to perform (e.g., in real-time) a tissue-based-omics analysis for a mechanism of action of a given study. The user can adjust the performance of the MTC by controlling the control system through the user interface as the study or experiment develops.

The systems and methods described herein enable modular arrangement of the MPSs on an MTC for emulating an immune system. Fluid media can be circulated and recirculated through the tissues of the MPSs for emulation of the immune response by a human. In some implementations, the MTC can test drug absorption and the response of the tissues to the introduction of the drug into the fluid media.

The MTCs described herein can include MPs that have basal and apical compartment to support basal and apical fluid loops. In an example, the MTC can include an inlet and an outlet to an apical compartment to introduce gaseous mixtures to the tissue and measure the response. As previously discussed with respect to other compositions like drugs, introduction of gases can be controlled in real-time with the controller, and sensor measurements can be received and processed in real-time. In an example, real-time sensor data can be used by the controller as feedback to control introduction of additional gaseous mixtures, drugs, or to control operation of (e.g., change or adjust) the circulation or recirculation of fluid media in the MTC as the study progresses.

The MTCs described herein can be used for particular example workflows. In a first example workflow, a toxidrome assessment is performed. In a second example workflow, a CBRN-injury and MCM testing study is performed. However, these are illustrative workflows and not exhaustive. The modularity and real-time control of the MTC enable countless different studies to be performed in different ways to suit the precise needs of the user.

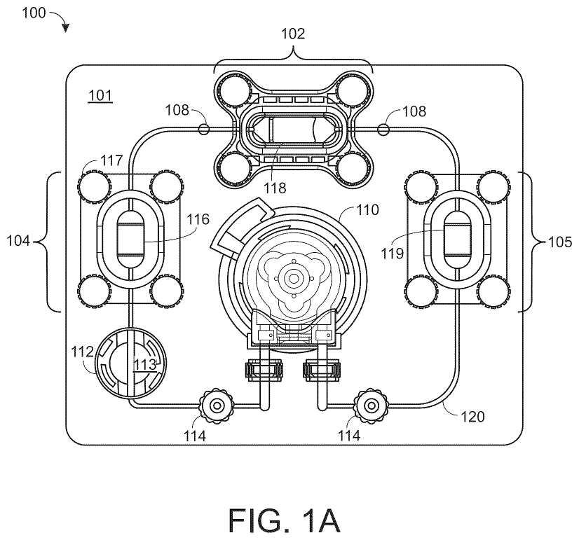

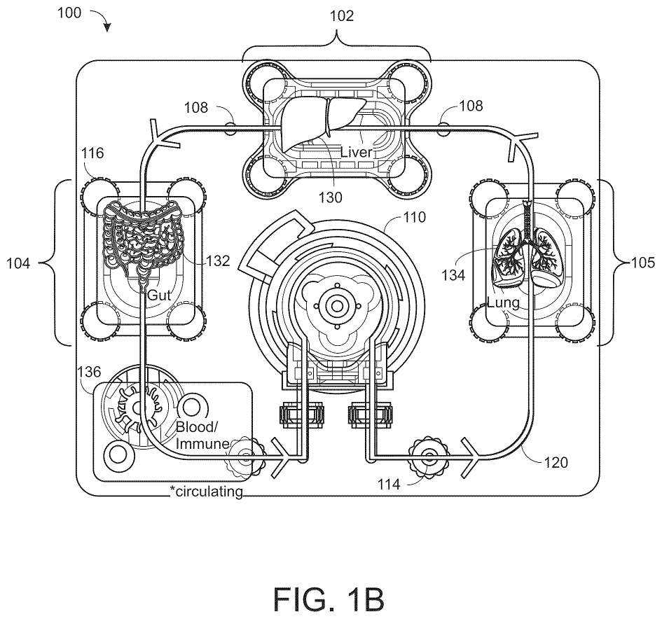

FIG. 1A is a diagram showing a top view of a multi-tissue chip (MTC) 100 including a multi-MPS microfluidic device. The MTC 100 can include up to three immunocompetent MPSs 102, 104, 105 with recirculating immune cells. MTC includes a recirculating fluid loop 120 that circulates a fluid media through the MPs 102, 104, 105. The fluid media can emulate blood flow and can carry oxygen to tissue in each of the MPSs 102, 104, 105. In an example, diseases (such as bacteria or viruses) or drugs can be added to the fluid media to test an immune response, recovery, and/or measure drug absorption in the tissues of the MPSs 102, 104, 105.

The MPSs 102, 104, 105 can be modular such that they are removable/replaceable on the MTC 100 for arranging different studies or emulating different fluid loops on the MTC. The MPSs 102, 104, 105 can be fastened to the MTC with fasteners 117. The MPS are standard modules that integrate with the fluid loop 120 when the MPS is fastened to a substrate 101 of the MTC. Each MPS 102, 104, 105 can be configured to host a particular tissue and can have a chamber design specific to that particular tissue. For example, the MPSs 102, 104, 105 can be configured to host one of liver tissue, brain tissue, lung tissue, gut tissue, spleen tissue, or other human tissue. The order of the circulation of the fluid media in the loop 120 being circulated through the MPSs is set based on the position of each of the MPSs 102, 104, 105 in the loop. In this example, 3 MPSs 102, 104, 105 are shown in a loop arrangement, but it is possible to include other numbers of MPSs in a modular configuration.

A pump 110 is configured to circulate and recirculate fluid media through the MPSs 102, 104, 105 of the MTC 100 in the fluid loop 120. The fluid loop 120 is defined by microfluidic channels that ensure a controlled flow rate of the fluid media through each of the MPSs 102, 104, 105. The pump 110 is controlled by a controller (described below) to ensure that the fluid media does not impart a shear force on the tissues hosted in the MPSs such that the tissues are damaged. In addition, the controller ensures that the fluid media are circulated or recirculated at a sufficient flow rate to ensure that the tissues are properly oxygenated. The flow rates and shear forces are described in WO Pub. 2023/023652, incorporated by reference herein in entirety. In an example, a chamber 116 of the MPS 104 can be configured to host spleen cells and a glucose sensor. In an example, a chamber 118 of the MPS 102 can be configured to host liver cells. The chamber 119 of MPS 105 can be configured to host lung or gut cells and include a TEER sensor for measuring barrier integrity, e.g., based on electrical signals.

The fluid loop 120 can include both apical and basal fluid loops. In some implementations, chambers 116, 118, 119 include both apical and basal portions to enable fluid flow on either side of a tissue membrane of a tissue hosted in a respective chamber. In some implementations, the fluid loop 120 has two separate sub loops. In some implementations, the fluid loop includes branches.

The controller is configured to detect the types of MPSs 102, 104, 105 and their arrangement in the MTC 100. The controller is configured to control the pump 110 to circulate the fluid media based on which MPSs are included in the fluid loop. In some implementations, circulation can be controlled based on the order of the MPSs.

Access ports 114 enable a user to introduce compositions to the fluid media for circulating through the MPSs 102, 104, 105. In an example, a drug can be introduced to the fluid media for measuring absorption by the tissues of the MPSs 102, 104, 105. In another example, a disease, as a virus or bacterial cells, can be introduced to the fluid media to measure an immune response by the cells of the tissues in the MPSs 102, 104, 105.

A sampling port 112 enables fluid media to be removed from the fluid loop 120. The sampling port 112 enables removal of fluid media without contamination of the fluid media that is being recirculated. The sampling port 112 is near an oxygenation chamber 113 that adds oxygen to the fluid media in the fluid loop 120 in a controlled amount as the fluid recirculates through the oxygenation chamber.

Sensors in the respective MPSs are configured to measure how the tissues respond to introduced compositions or what a status of the tissues are as fluid media is circulated in the fluid loop 120. The sensors can be positioned in the fluid loop 120, such as on either side of a given MPS. For example, oxygen sensors 108 are on either side of MPS 102 to measure oxygen absorption in tissue of the MPS 102 (e.g., liver tissue). While oxygen sensors 108 are shown near MPS 102, the oxygen sensors can be positioned in additional or substitute locations in the MTC 100.

Alternatively or in addition to sensors placed in the fluid loop 120, sensors can be integrated with the modular MPSs 102, 104, 105. The selection of sensors can be based on the type of tissue being hosted in the cell chambers 116, 118, 119 of the MPSs 102, 104, 105. For example, TEER sensors can be used to measure barrier integrity for gut tissue or lung tissue. Oxygen sensors can be integrated with an MPS to measure oxygen absorption, such as by liver tissue. Imaging sensors can be placed near the oxygenation chamber 113 to measure content of the blood or fluid media being circulated in the fluid loop 120. Further sensors and configurations are described herein.

The MTC 100 is configured to host human cells including primary or iPSC cells. The MTC 100 enables long-term functionality of the cells once introduced to the MPSs to run studies of multiple weeks (e.g., over 30 days). The MTC enables relatively high content of cells, the chambers are sized to host large tissue samples with millions of cells. The MTC 100 can measure media and tissue biomarkers and perform imaging and sampling throughout a study without pausing the study or disrupting media circulation. The control system, described in further detail below, allows a user, through a user interface, to control the study in real-time and observe predictive and translational results for drug discovery applications.

In an aspect, the MTC 100 is configured for modular MPSs 102, 104, 105 to conduct CBRN threat assessment & MCM development. The MTC 100 is configured for integrated MPS types such that any arrangement of MPSs from the library of available MPS types can be used. For example, an MPS with a particular geometry that is suitable for a respective type of tissue can be obtained, and the tissue can be seeded on the MPS. Once the tissue is ready, the MPS can be added to the fluid loop 120 for performing a customized study.

The MTC 100 can be configured to perform studies of threat exposure routes, studies of chemical medical countermeasures (chemical MCM) administration routes, perform real-time multiplexed on-chip biosensing, perform CBRN threat detection, measure and/or monitor tissue health, monitor immune response, and operate as an autologous multi-MPS system for physiologically-relevant immunology studies, The MTC 100 is configured to generate predictions for humans based on measurements of clinical biomarkers, emulate first-in-human (FIH) dosing, perform population analysis, analyze genome-scale metabolic networks, and perform compound target identification for development of drugs.

The MTC 100 is configured to perform high throughout assay analysis. A plurality of the MTCs can operate in parallel and be controlled by a single controller for a given study. In some implementations, 2, 4, 8, 16, or more instances of the MTC 100 can be controlled in parallel. In some implementations, dozes or hundreds of MTCs 100 can be controlled together for a given study.

FIG. 1B is a diagram showing a top view of an example of the MTC 100 of FIG. 1A including example tissues 130, 132, 134, 136 for measuring immune responses or performing the studies described herein. MPSs 104, 105 can be integrated with TEER sensors for measuring barrier integrity for a barrier-based MPS, such as lung, skin, or gut MPSs. To test a route of exposure, MPS 102 can include oxygen sensors 108 for high-consumptions tissues (e.g., liver tissues) to assess liver-specific adverse effects of mitochondrial toxicity caused by CBRN threats to be assessed, as well as metabolism and bio-transformations. In some implementations, the MPS 104 can include a glucose or lactate sensor for immune assessments (e.g., T cell activation) to evaluate adverse outcomes of CBRN threats at a target tissue. The example MTC 100 shows three immuno-competent MPSs 102, 104, 105 that are fluidically linked by a recirculating common medium loop 120 with circulating immune cells for thorough assessment of CBRN threats.

The MTC 100 shows a gut MPS 132, a liver MPS 130, and a lung MPS 134. However, other combinations of tissues, including different tissue types and orders, are possible, as described herein. The MTC 100 includes imagine sensors to measure blood or immune response from immune cells 136.

The MTC 100 of FIG. 1B is configured for studying iPSC-derived autologous immune response for a gut-lung-liver-blood arrangement. In some implementations, an aptamer-based multiplexed sensor is included. Specifically, these include immuno-competent lung issue, immuno-competent liver tissue, immuno-competent gut tissue, and measurements of circulating blood/immune cells. In some implementations, the sensors include aptamer- based proteomic sensors. The aptamer-based proteomic sensors are configured for biomarkers discovery based on simultaneously measuring thousands of proteins from small sample volumes (e.g., micro-liters). The MTC 100 is configured for real-time biosensing of pathogen exposure and adaptive & innate immune responses. The MTC 100 can be used to assess responses to airborne or oral chemical & biological threats and, simultaneously, countermeasure therapies (mAbs, small molecules, vaccines) via oral, IV, and inhalation routes.

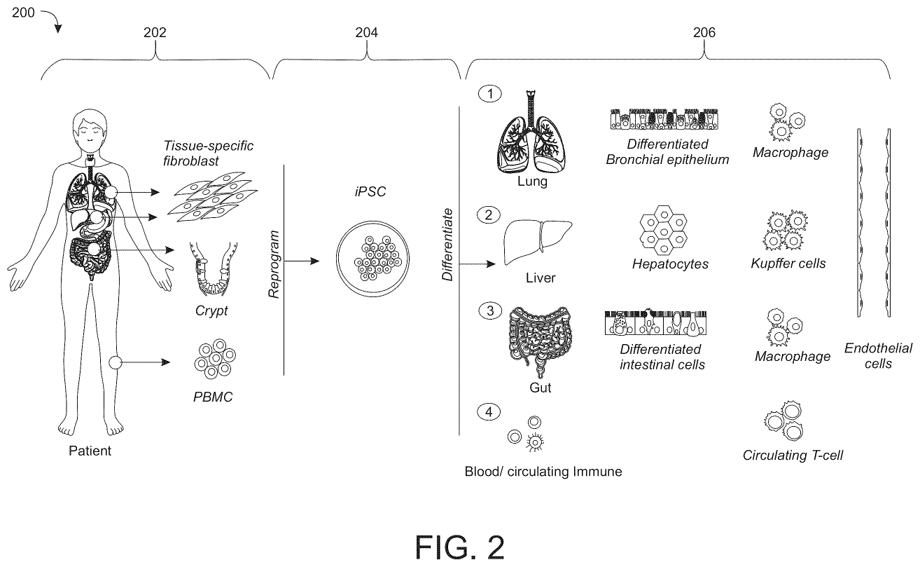

FIG. 2 is a diagram showing an example process 200 for measuring immune responses. The process 200 is configured to use autologous iPSC tissue with a donor-matched immune system. Specifically, tissues are obtained (202) from a donor for various tissue types (e.g., tissue-specific fibroblasts). iPSC cells are incubated (204) based on the donor cells. The iPSC process is highly scalable and provides consistent resources for multiple cell types. From the iPSC cells, the system generates (206) immuno-competent lung issue, immuno-competent liver tissue, immuno-competent gut tissue, and measurements of circulating blood/immune cells (or other such cells) for including in respective MPS modules previously described. For example, the cells can include endothelial cells, differentiated bronchial epithelium cells or macrophage cells, hepatocytes or Kupffer cells, differentiated intestinal cells or macrophages, circulating T-cells, and so forth.

Workflows for using the MTC 100 are as now described. In a first workflow, a use can expose the cells to chemical of concern. In a first step, an exposure is emulated. The exposure can be systemic or inhalation, in an example. In a second step, the MTC 100 circulates the fluid media and monitors the MPS-specific responses in real-time. As described previously, the analysis can include continuously or semi-continuously measuring cell parameters or blood content from the sensors integrated with the respective selected modular MPS of the MTC 100. These sensors send data to the controller which respectively presents the data to the user in a user interface or stores the data for later analysis. In some implementations, the analysis can be performed in an ongoing basis. The analysis can include mechanism of action (MoA) analysis for a drug discovery process,-omics and systems biology analysis, and so forth, depending on the particular study. The process includes identifying affected MPSs for toxidromes. A toxic syndrome or toxidrome is a ‘clinical fingerprint’, characterized by a classic constellation of symptoms and signs due to toxic effects of chemicals in the body. The toxidromes can include pulmonary, neurological, metabolic, or other toxidromes, depending on the particular MPS that is selected. In an example, an MTC 100 can be used to analyze around 12-15 CBRN threats for three toxidromes.

An example study is now described to develop a phosgene-induced lung injury model. In this example, the MTC 100 is used to detect a primary injury including cytotoxicity or barrier damage. In this example, the MTC 100 is used to detect a secondary injury such as inflammation & immune cell recruitment. Once the MTC 100 is established with the appropriate MPSs (e.g., brain, liver, and lung MPSs), the MCM is administered (e.g., by emulation of IV or inhalation). A prophylactic or post-threat exposure is measured. In some implementations, the study is configured for systemic, aerosol inhalation. The study can include either single and/or combination therapies and analyze the results on the tissues. The controller, via the data from sensors, is configured to monitor the efficacy of MCM in real-time or near real-time. In an example, the MTC 100 can evaluate ˜20 drugs with various MoAs as mono and/or combination therapies.

Example drugs for MCMs are now described. Drugs can be configured for targeting a primary injury. These drugs can include oxidative damage and antioxidants such as N-acetylcysteine (NAC), Glutathione, AEOL 10150, and SS31. Other such drugs are possible for inclusion.

These drugs can include inflammation and anti-inflammatory drugs such as dexamethasone, prednisone, and ibuprofen. Other such drugs are possible for inclusion.

These drugs can include fibrosis and anti-fibrotic drugs such as Pirfenidone, Nintedanib, and Colchicine. Other such drugs are possible.

These drugs can include drugs targeting secondary injury. These drugs can include Neutrophil recruitment & neutralizing mAbs, such as anti-IL8 mAbs and anti-IL1B mAbs. Other such drugs are possible for inclusion. These drugs can include vascular injury and phosphodiesterase inhibitors such as CP-80633, Sildenafil, and Theophylline. Other such drugs are possible for inclusion. These drugs can include transient receptor potential (TRP) cation channel inhibitors such as SKF 96365 and A-967079. Other such drugs are possible for inclusion. These drugs can include additional drugs targeting various mechanisms in lung injury. These drugs can include Simvastatin, Metformin, Vigabatrin, Valproic Acid, Captopril, and Ambrisentan. Other such drugs are possible for inclusion.

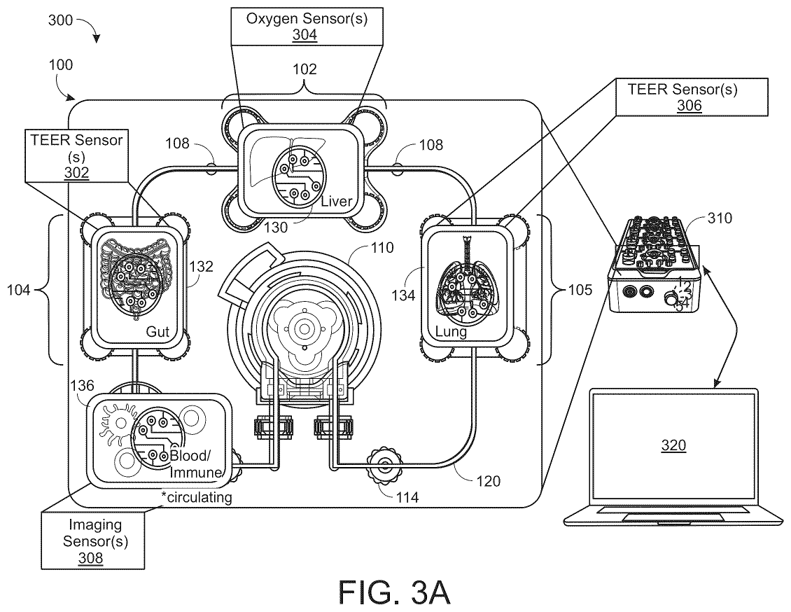

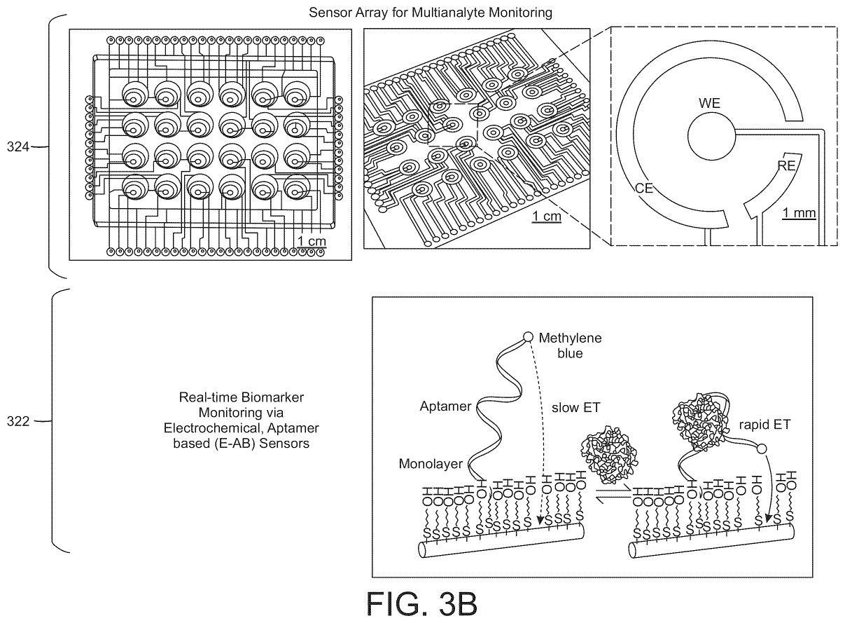

FIG. 3A is a diagram showing an example system 300 including the MTC 100 of FIGS. 1A-1B. The system 300 is configured for modular aptamer-based multiplex sensors and tissue health real-time monitoring. The system 300 can include 5-plex aptamer-based sensors having <50 mm² footprint. The system 300 is configured for the following uses: CBRN-threat detection (e.g. spike protein); MPS-specific response (e.g. liver CRP); Immune response-markers (e.g. IL-6, & glucose/lactate); and adverse effects (e.g. LDH). The system 300 is cnf for tissue health monitoring. TEER sensors 302, 306 can be included for measuring barrier integrity for gut & lung MPSs 132, 134. Oxygen sensors 304 can measure oxygen levels, such as for the liver MPS 130. Real-time imaging sensor(s) 308 can be included for analysis of blood or immune cells in the MPS 136.

The system 300 is configured to assess biological responses. For example, the system 300 can perform real-time monitoring for airborne viral pathogens through the lung MPS 134 (e.g. SARS-CoV2). The system 300 can perform real-time monitoring for oral viral pathogens through the gut MPS 132 (e.g. rotavirus). The system 300 can perform real-time monitoring of an MPS response for each pathogen.

The system 300 can evaluate clinical therapies. Characterization studies on known approved and unapproved therapeutic candidates can be performed, such as for Dexamethsone, Bamlanivimab (anti-spike protein), Casirivimab and Imdevimab (anti-spike protein), Baricitinib (JAK inhibitor), or Sotrovimab (anti-spike protein). An administration route can include oral, IV, or inhalation emulation. The system 300 can evaluate MCM metabolism, dosing strategy, and efficacy to demonstrate agreement with established preclinical and clinical data.

The system 300 includes the MTC 100, a controller 310, and a client device 320 for rendering of a user interface. A user, through the client device 320, can control execution of the study by controlling media circulation through one or more instances of the MTC 100. As described in further detail below, a user interface enables the user to configure the analysis being performed, indicate the presence and/or configuration of MPSs in the MTC 100 (e.g., type and order of the MPSs), control operation of the sensors on the MTC 100 (e.g., manage sensor measurement frequency, precision, etc.), start or terminate the analysis, select data types or models for analysis, configure how analysis output affects subsequent control of the circulation of the fluid media via pump 110 and operation of the sensors of the MTC 100, and so forth.

The controller 310 includes a platform that for hosting a plurality of MTC instances. For example, the controller 310 can support up to 2, 4, 8, 16, or another number of MTC instances. The controller 310 supports an MTC instance by sending data to and receiving data from the sensors of the MTC instance and sending these data to and receiving control data from the client device 320. The controller 310 supports the MTC instance by receiving control data from the client device 320 for changing operation of the MTC, such as for changing circulation of the fluid media in the MTC fluid loop 120. The controller 310 can be configured to read sensor data responsive to receiving a control signal from the client device 320. The sensor data can be sent to the client device 320 and displayed on the user interface in real-time either continuously or in real-time responsive to a request for data from the client device 320.

FIG. 3B shows examples of sensors for being used with the system of FIG. 3A. The sensors are configured for real-time biomarker monitoring via electrochemical, aptamer-based (E-AB) sensors 322. Electrochemical aptamer-based (E-AB) biosensors are configured to generate an electrochemical signal in response to specific target binding in vivo. The signal can be measured by a change in Faradaic current passed through an electrode. E-AB sensors are configured for detecting target binding in vivo with real-time measurements. An E-AB sensor can include a three-electrode cell: an interrogating electrode, a reference electrode, and a counter electrode. A signal is generated within the electrochemical cell then measured and analyzed by a potentiostat. Several biochemical and electrochemical parameters optimize signal gain for E-AB biosensors. The density packing of DNA or RNA aptamers, the ACV frequency administered by the potentiostat, and the chemistry of the self-assembling monolayer (SAM) are all factors that determine signal gain as well as the signal to noise ratio of target binding. The E-AB biosensor can provide to the controller data representing in-situ sensing, feedback-controlled drug administration, and cancer biomarkers. In some implementations, the sensors can include aptamer-based sensors such as fluorescence generating aptamers.

In some implementations, sensors can include a sensor array 324 for multianalyte monitoring. The sensors can include enzyme-based multi-analyte biosensors. The sensors can be configured to monitor lactate and glucose through amperometry. In an example, the sensors can include working electrodes, a counter electrode, and a reference electrode. The sensor can be placed directly into a substrate that hosts the tissue. The electrodes can be functionalized, such as with oxidase enzymes, to enable continuous monitoring of lactate and glucose through amperometry.

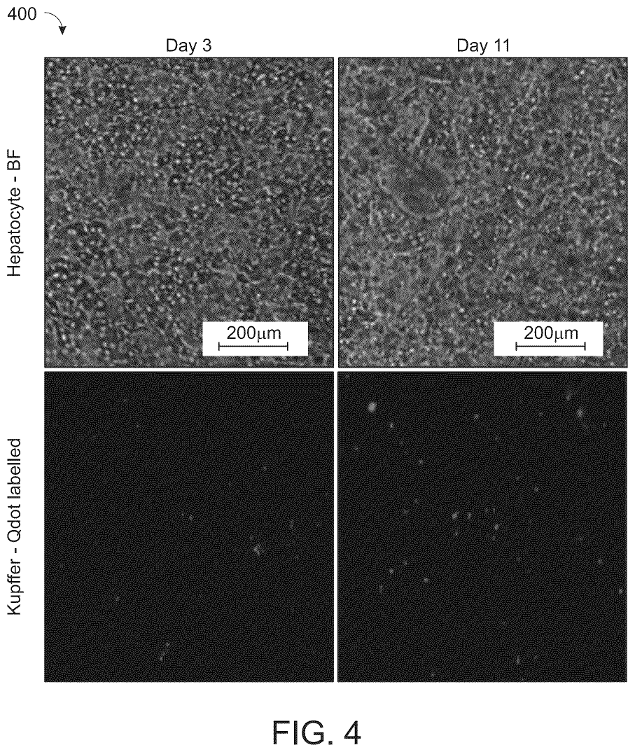

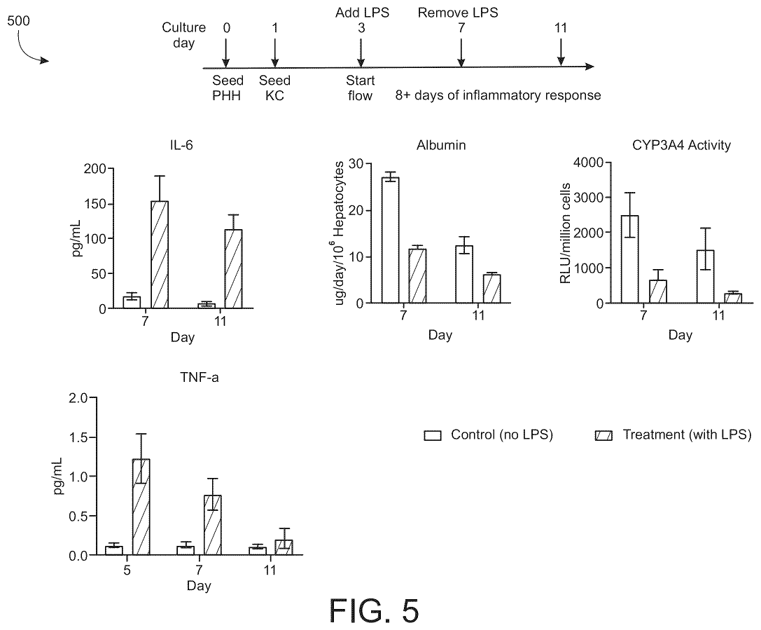

FIG. 4 shows example data 400 representing a chronic liver inflammatory response on a multi-MPS microfluidic device. FIG. 5 shows example data 500 representing a chronic liver inflammatory response on an MTC. These data are example data generated by operation of an MTC such as MTC 100. These data show a sustained inflammatory response of a liver co-culture MPS to LPS treatment, including decreased albumin levels, decreased CYP3A4 activity, and increased IL-6, TNF-a production.

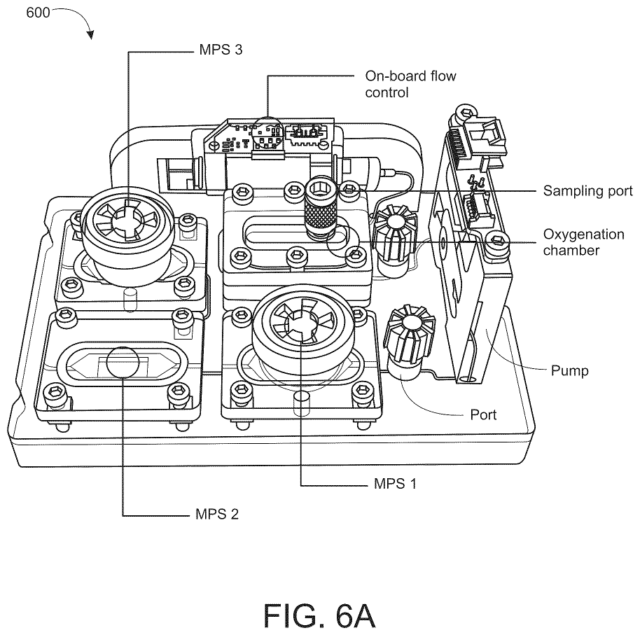

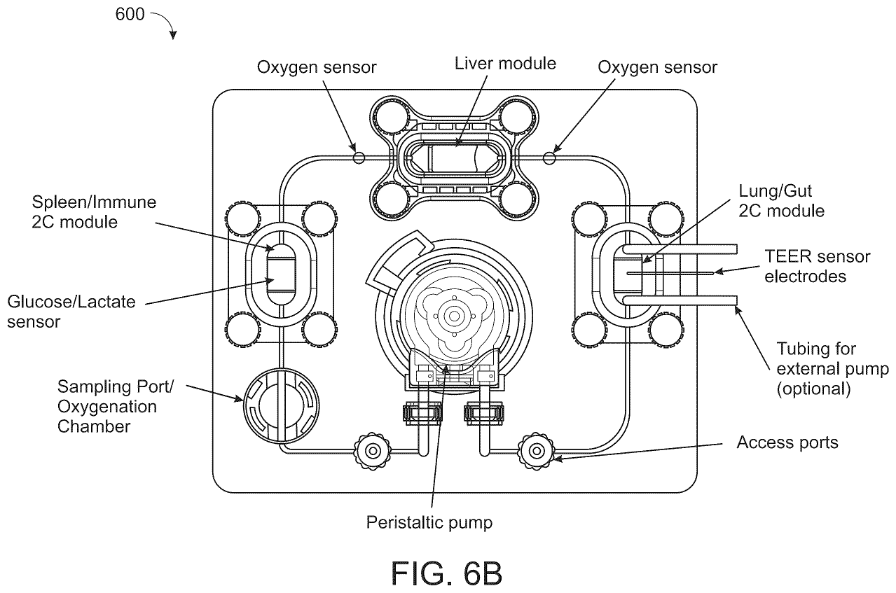

FIG. 6A shows a perspective view of an example of an MTC 600, which can be similar to MTC 100. The MTC 600 is at a milli-fluidic scale, having relatively large tissue sizes media volumes. The MTC 600 is configured for repeated media sampling from basal recirculation and apical compartments to evaluate TK and TD. The MTC 600 hosts a sufficient cellular mass for tissue analysis for (e.g. —omics). The MTC 600 is configured for basal recirculation in the fluid loop, such as fluid loop 120. As previously discussed, the MTC 600 is configured for modular tissue integration, in this case, up to 3 MPSs. The material of the MTC 600 is configured for low, non-specific binding, as it is comprised of Cyclic olefin copolymer (COC) plastic. The MPSs are each compatible with sensor integration, as previously discussed, including oxygen sensors, TEER sensors, and sensor arrays for multianalyte monitoring.

FIG. 6B is a diagram showing a top view of an example of the MTC 600 of FIG. 6A. A cytoarchitecture of immune-competent MPSs of the MTC 600 is shown. These are integrated on the MTC 600. A Lung MPS includes normal human bronchial epithelial cells (NHBEC) on top of hydrogel-embedded human lung fibroblasts cultured at air-liquid interface in the apical compartment with human lung microvascular endothelial cells on the basal side of the membrane. A gut MPS includes primary small-intestinal epithelial cells expanded as organoids and seeded as monolayers with CD45+ tissue-resident immune cells from the intestinal lamina propria. A liver MPS includes a monolayer primary human hepatocyte (PHH) overlayed with ECM gel-embedded stellate cells and LSECs and Kupffer cells on top (6:1 ratio). A spleen MPS includes Isolated CD45+ tissue-resident immune cells from spleen red and white pulp (macrophages, dendritic cells, neutrophils, T and B lymphocytes) embedded in collagen I droplets. A circulating Immune/Blood MPS includes Ficoll purified peripheral blood mononuclear cells (PBMCs).

The MTC 600 includes a milli-fluidic format, incorporating both a large MPS size and media volume, allows repeated media sampling from basal recirculation and apical compartments to evaluate exposure-response relationships and-omics analysis to investigate secreted biomarkers. The design also provides sufficient cellular mass for tissue-based-omics analysis for mechanism of action of CBRN threats.

A basal recirculation allows analysis of circulating tissue-secreted molecules (e.g., cytokines), and assessment of hepatic clearance of the CBRN threats. In contrast, an MPS with a traditional flow-through design obscures the ability to detect concentration changes over time.

Modular tissue and sensor integration combines a plurality of MPSs (e.g., three) and respective sensors to be combined in a modular fashion to ensure that the MTC 600 can be configured for the specific threat encountered. MPS types that can be combined include liver, lung, gut, skin, cardiac, spleen, and more. Table 1 shows examples of sensors for monitoring in the MTC 600 (or MTC 100 described previously).

TABLE 1
Real-Time Monitoring of Tissue Health and Functionality

| Analyte | Readout | Method |
|---|---|---|
| IL-6; IL1B | inflammation | aptamer-based sensor |
| Albumin | liver function | aptamer-based sensor |
| Acetylcholinesterase; | neural function | aptamer-based sensor |
| N-acetylaspartate (NAA) | | |
| Glucose | metabolism | aptamer-based sensor |
| Lactate | metabolism | aptamer-based sensor |
| TEER, lung | lung function | electrode electrical resistance |
| TEER, BBB | BBB function | electrode electrical resistance |
| Real-time Imaging | BF/Fluorescent | integrated microscope |

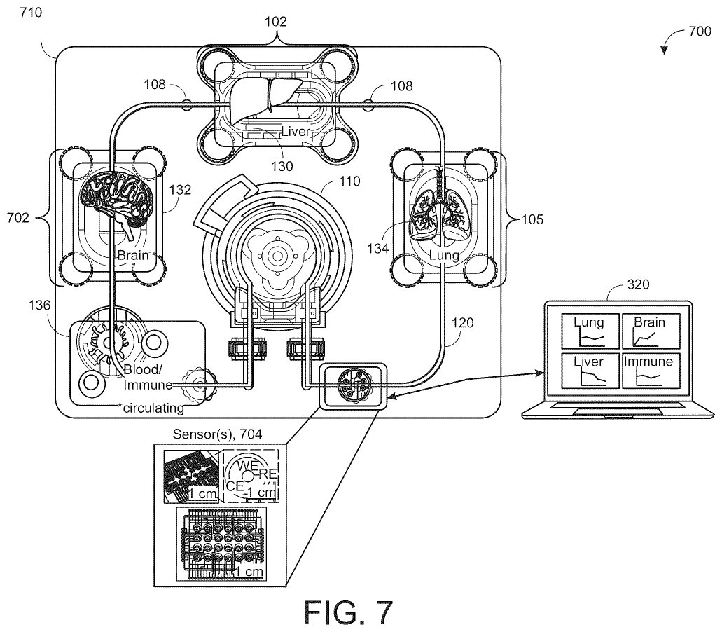

FIG. 7 is a diagram of a system 700 including an example of an MTC 710. MTC 710 is similar to MTC 100 except that MTC 710 includes brain tissue in MPS 132. The MTC 710 functions as a multi-tissue chip platform interconnecting a Lung-Liver-Brain-Blood MPS with aptamer-based multiplexed and real-time biosensing for toxidrome risk assessment of emerging CBRN threats. The MTC 710 is used for development of CBRN-injury MPS models and for efficacy testing of medical countermeasures.

In an example, each MPS will have parenchymal, resident immune and vascular components. The Lung MPS includes Pneumocytes, Macrophages and epithelial cell types (EC). The liver MPS includes PHH, KC, LSEC cell types. The brain MPS includes Neurons, Microglia, EC cell types. The blood/immune MPS includes circulating immune cells. The MTC 710 is configured for multi-MPS integration for a common medium development for integrated lung-liver-brain-blood MPSs.

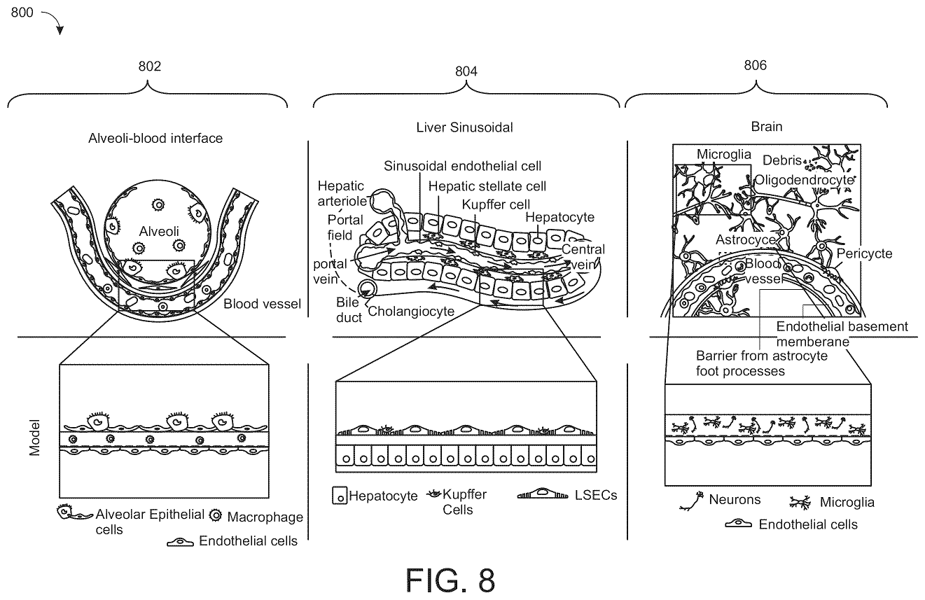

FIG. 8 shows examples of models for testing immune responses in tissues, such as for MPS 710 of system 700. The models can include a alveoli-blood interface model 802, a liver sinusoidal model 804, and/or a brain model 806.

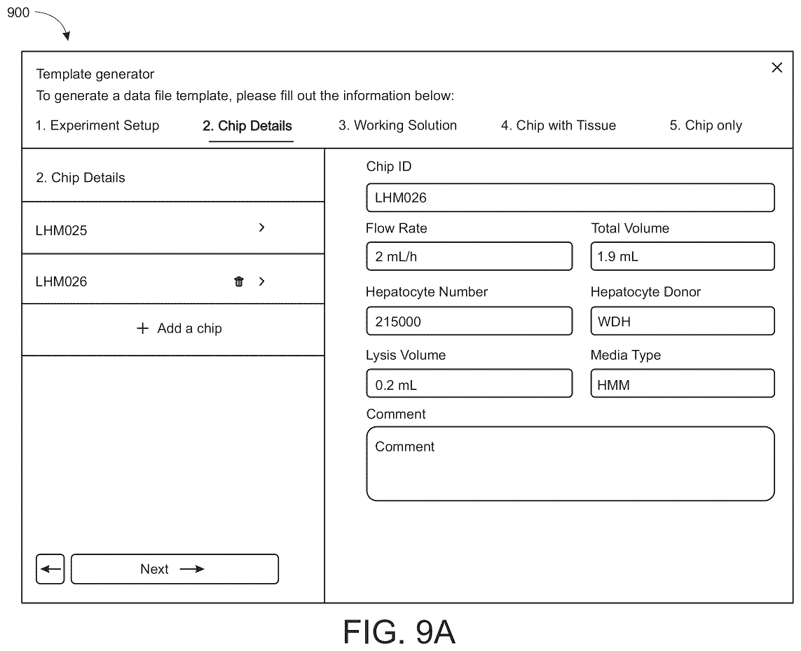

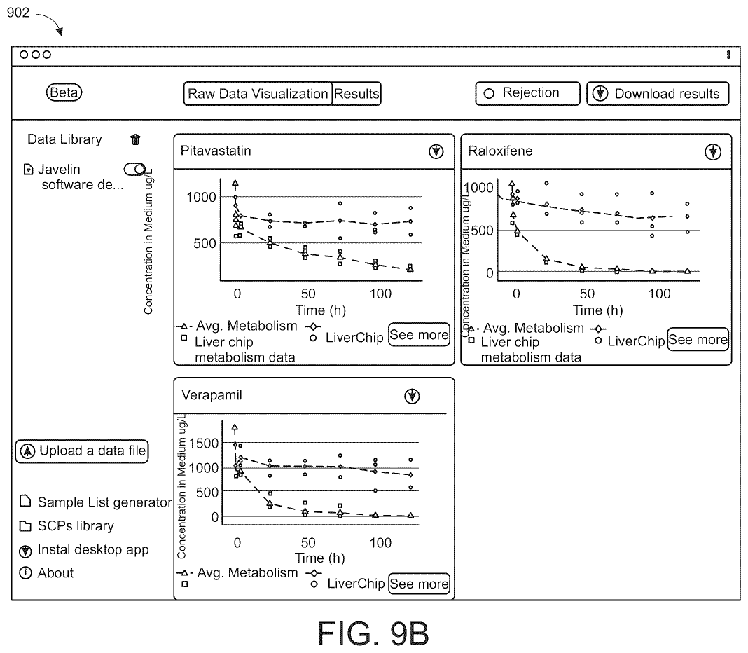

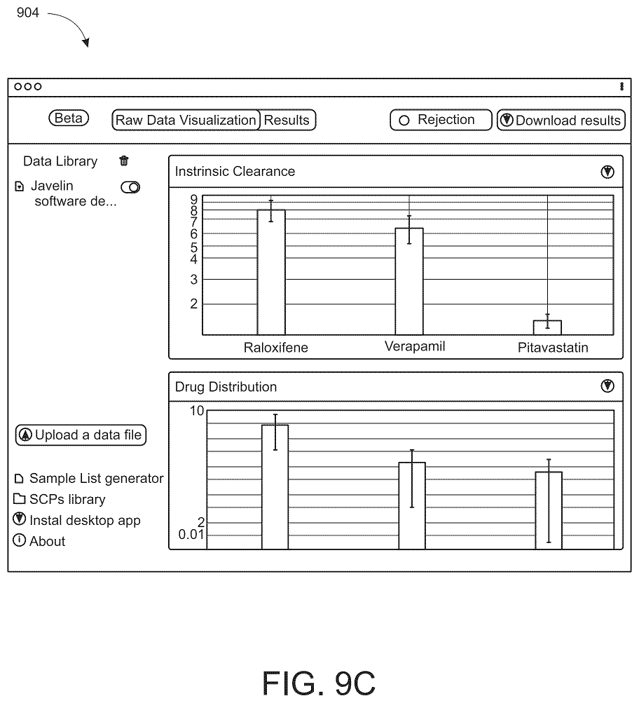

FIGS. 9A-9C show examples of user interfaces 900, 910, 920. The user interfaces 900, 910, 920 streamline and standardize sample generation and data analysis from MPSs of the MTC. The user interfaces can be used to specify on-chip and in vivo parameters for prediction of values and enable the predictions to occur in real-time. The user interfaces 900, 910, 920 are designed to enable non-technical users to configure the hardware and set up a test that may otherwise require custom hardware integration.

In an example, interface 900 enables a user to specify MTC chip details. The user can indicate a chip identifier, specify a desired flow rate (e.g., 2 mL/hour), specify a total volume of flow of fluid media, specify tissue type and size, specify a donor identifier, specify a type of fluid media, and so forth. The entire analysis is configurable by the user prior to and during execution.

Interface 902 enables a user to view analysis results in real-time as the analysis is being performed. The user can view measurements drug levels/absorption, immune response, cell activity, concentrations of disease or other CBRN aspects, as so forth. The user can view these measurements in real-time and adjust the operation of the MPSs of the MTC during execution of the analysis.

Interface 904 shows examples of measurements from the fluid loop of an MPS. The charts show drug distribution and intrinsic clearance values over time for each MPS. While these example data are shown for illustrative purposes, any sensor data or predictions from the sensor data can be presented to the user through one or more of interfaces 900, 902, 904.

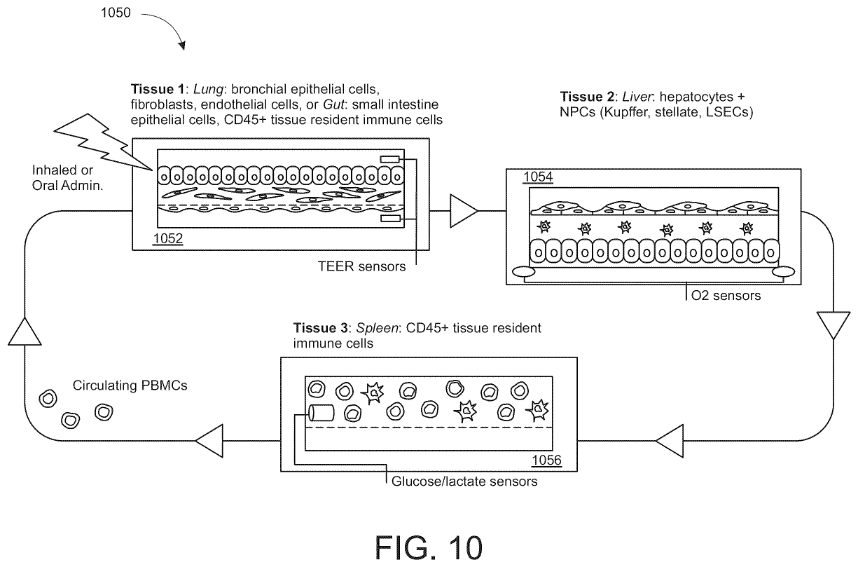

FIG. 10 shows an example of testing an immune response using an MTC 1050. The MTC 1050 enables long-term MPS culture from primary human cells. The MTC 1050 is characterized for integrated tissue functionality and pharmacokinetic applications. Combined with the integrated sensors, this allows continuous monitoring for more than 30 days, as described previously. MTC 1050 can be similar to MTCs 100, 710 previously described.

The MTC 1050 has low non-specific binding. The MTC 1050 can be manufactured with thermoplastics (COC), which lowers non-specific binding. The MTC 1050 is compatible for pharmacology applications, sensor integrations and mass production.

Two use-cases demonstrate platform modularity. Lung-liver-spleen (non-donor match) MPSs 1052, 1054, 1056 are integrated to study tissue-specific responses to inhaled chemical threats, such as organophosphates. Primary human cells are obtained from commercial sources to show that the MTC can be used with readily available material and established protocols to assess inhaled CBRN threats. A lack of donor-matched models capable of local and systemic immune responses is overcome. To accurately predict human immune responses in vitro, MPSs and immune cell populations are derived from the same individual to prevent allograft rejection. Furthermore, it is not enough to include only circulating PBMCs since each integrated tissue needs to be accompanied by its own tissue-resident immune cell pool to mount accurate local immune responses. MPS models are formed to create immunocompetent multi-tissue model. A donor-matched model of the gut-liver-spleen axis is modeled. The model incorporates: tissue-resident immune cell populations found in the gut mucosa and liver; circulating blood-derived immune cells (PBMCs); and a spleen that serves as a secondary lymph node organ capable of facilitating antigen presentation and T-cell effector expansion.

In another example, an integrated liver-kidney-muscle MPS on an example MTC can be built with a pharmacokinetic context of use (CoU) evaluation. The MTC can be formed from thermoplastic COC, which has minimal non-specific binding to lipophilic drugs. The integration of human primary liver MPS (hepatocytes), human primary kidney MPS (RPTECs) and human primary muscle MPS (skeletal muscle) to maintain viability and functionality.

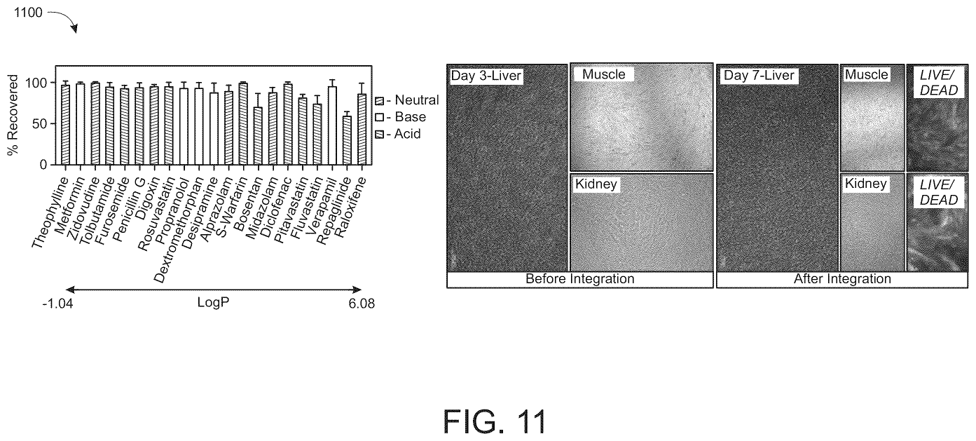

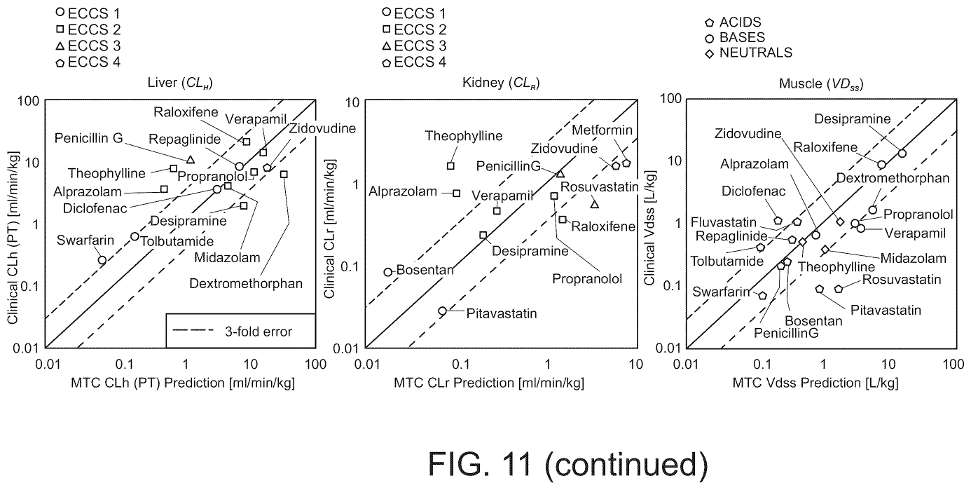

FIG. 11 shows data 1100 representing a pharmacokinetic CoU evaluation with small molecule drugs. The MTC can accurately predict three clinical PK parameters (hepatic (CLH) and renal (CLR) clearance and volume of distribution (VDss).

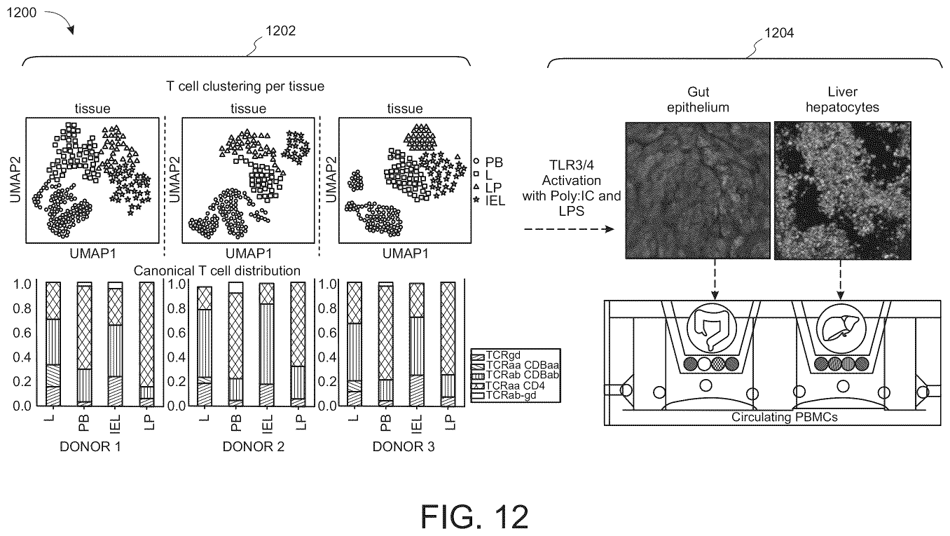

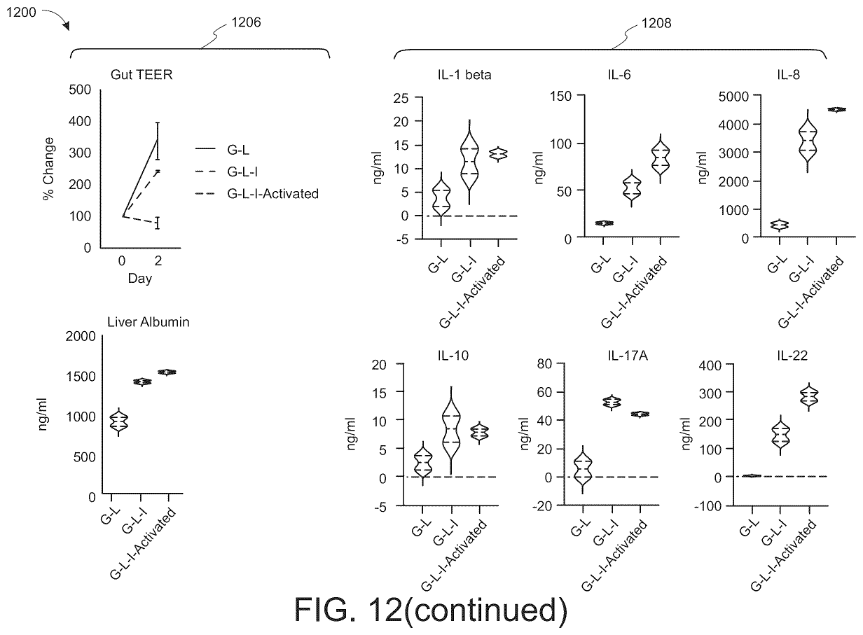

FIG. 12 shows a donor-matched model 1200 output of a gut-liver axis. A single-cell RNA sequencing characterization of 3 donor-matched blood, gut and liver tissue is shown at 1202. On top, T cell clustering per tissue is shown, including PBMCS (PB), liver (L), and gut lamina propria (LP) and intraepithelial space (IEL). On the bottom, a distribution of major T cell subtypes per tissue is shown including CD4 T cells, CD8 T cells, and gamma-delta T cells. Section 1204 shows a schematic representation of gut-liver interaction studies where gut and liver tissue are co-cultured with their own tissue-resident immune cells and perfused with universal media containing donor-matched PBMCS. The interacting system was challenged with Poly:IC mimicking viral infection. Section 1206 shows a gut barrier function compared between gut-liver interaction in absence of immune cells (G-L), with immune cells (G-L-I) and when challenged (G-L-I-Activated) on top. On the bottom, liver albumin production data compare the three conditions. A section 1206, significant induction of inflammatory cytokines are detected in common media as a result of an intestinal challenge with Poly: IC.

In another example, an MTC including of liver, kidney, and skeletal muscle MPS is generated for pharmacokinetic (PK) studies. In these studies, a diverse set of small molecule drugs from all extended clearance classification system (ECCS) classes with various clearance mechanisms was evaluated. On-chip PK parameters were quantified, such as hepatic metabolism, uptake & disposition, tubular secretion & reabsorption, and muscle disposition and, then computationally scaled those to clinical parameters for IV drugs: hepatic clearance (CLh), renal clearance (CLr) and volume of distribution (Vd). The predicted PK parameters showed high correlation to clinical parameters. The data demonstrated that the human MTC platform could deliver accurate PK parameter predictions and were applicable for mixed clearance mechanisms and drug-drug interaction studies (data not shown). In addition, human primary same donor gut-liver-spleen MPS were developed, integrated with recirculating PBMCs, and characterized extensively for their immune functionality.

The MPSs described herein ensure a robust and sustainable product pipeline for MCMs that emphasizes multi-functional capabilities rather than stand-alone outcomes (e.g., platform technologies, host-based innovations, broad-spectrum MCMs), and includes consideration of viable commercial markets and/or routine public health applicability.” The MPS is configured to perform functions for at least five components: 1) infection with a relevant viral, bacterial, or fungal pathogen as well as insult with toxins or toxicants; 2) integration of at least two different tissues in addition to the immune component(s); 3) near-continuous monitoring of the MPS for at least two weeks (using three sensors); 4) (semi-) automated manufacturing of the platform; and 5) biological characterization of the MPS and recapitulation of existing clinical data in response to immune responses to chemical and biological threats.

In an example embodiment, the MTC is updated for immunology and CBRN threat assessment. This includes incorporation of three sensors for real time sensing of each tissue, MPS chamber and fluidic path modifications for immunology applications, switching to mass production compatible manufacturing methods, and optimization of manufacturability cost. In an example embodiment, the MTC is designed for CBRN applications and immune engineering and bio-sensing & software development.

Examples of MPSs are now described. A liver MPS can include a liver Quad-culture of primary human hepatocytes (PHH) and liver non-parenchymal cells (NPC: LSECs, Kupffer and stellate). The ECM coated liver culture chamber can be first seeded with PHH as a monolayer and overlayed with MatriMix hydrogel, a fully defined ECM substitute, which encapsulates stellate cells. After gelation of the hydrogel, LSECs and Kupffer cell mixture (6:1 cell ratio) are seeded on top of the ECM.

The lung MPS includes a lung airway. The lung MPS includes of normal human bronchial epithelial cells (NHBEC) and human lung fibroblasts apically and endothelial cells on the basal side of a porous membrane. In the apical side, first the fibroblast is embedded in a hydrogel composed of thiolated hyaluronic acid and methacrylated gelatin. The hydrogel is crossed linked using UV light and NHBEC are seeded on top of the gel. After several days of culture under both apical and basal media flow, the apical NHBEC are allowed to mature under airflow. After 7-10 days of culture, human lung microvascular endothelial cells will be seeded on basal side and is matured under flow.

A spleen MPS includes a donor-matched immuno-competent gut-liver-spleen MPS. The spleen MPS is developed to evaluate exposure-response relationships. The spleen MPS overcomes an obstacle for MTC that includes a lack of donor-matched models capable of local and systemic immune responses. A donor-matched, multi-MPS model for the studies of host-pathogen interactions solves this problem. The MPS addresses a gap in the prediction of microbial pathogenicity and provides means for therapeutic development. An immunocompetent three MPS is constructed and validated from same-donor tissue. The systems determine acute physiological responses to viral exposure and investigate toxin responses. The comparison of results with clinical ranges and established data enhances the translational potential of research and contributes to the development of novel therapeutic strategies and vaccines. This provides a mechanism for human preclinical modeling and pioneering preparedness against new pathogenic threats.

To establishing a biobank of donor-matched tissues, a portion of blood, gut, spleen, and liver tissue is stored unaltered. Both gut and liver tissues are enzymatically dissociated to generate liver and gut organoids. Organoids are grown based on established protocols in stem cell-factor enriched media and Matrigel. At the same time, tissue-resident immune cell populations are being sorted and stored for interaction studies. Spleen tissue is processed, and red blood cells lysed.

To establishing individual MPSs, the following steps can be performed. For an individual gut MPS, small intestinal cells can be cultured as organoids followed by seeding 100 k individual cells in monolayer format. Once mature, intraepithelial immune cells are added to the monolayers and placed on top of 20 k tissue-resident immune cells isolated from the gut lamina propria, encompassing a diverse pool of antigen-presenting cells and lymphocytes. Immune cells are thawed one day prior to experimentation and rested in Immunocult media. Performance criteria: TEER, FITC-dextran permeability, IL-10 and IL-33 production.

For an individual liver MPS, hepatocytes are cultured as organoids. Once matured, 600 k are seeded on a Matrigel substrate and covered with collagen I. In a sandwich format, 30 k liver-resident immune cells are added composed of Kupffer cells and diverse lymphocyte populations. Tissue resident immune cells are thawed one day prior to experimentation and rested in Immunocult media. Performance criteria: Production of Albumin, Urea, CRP.

For an individual spleen MPS, a processed spleen undergoes immune cell purification via cell sorting. 800 k isolated cells are embedded within a mix of collagen I and III and placed in drop shape within its MPS compartment. Performance criteria include Live/dead, production of IL-6, IL-10, 11-12.

To generate circulating PBMCs, the PBMCS are isolated from whole blood at the time of arrival in the lab. PBMCs are thawed one day prior to experiments and rested in Immunocult media overnight. On the day of integration, 100 k PBMCs per ml are added to the universal media that fluidically connects all individual compartments. Performance criteria: Live/dead, adequate responses after stimulation.

There are several types of use cases. In an example, the MPSs are used to assess chemical threats using organophosphates (OPs), which have a similar chemical structure to nerve gases. In another example, a biological threat assessment is performed using Ebola Virus-Like Particle and Toxic Shock Syndrome Toxin-1 (EVLP & TSST-1) molecules, which trigger similar immune responses to Ebola virus bacterial toxins. In each use cases, we will monitor MPS health with sensors (lung/gut barrier integrity, liver oxygen consumption rate (OCR), and glucose/lactate for T cell activation), daily samples collected from MTC medium for secreted biomarkers (e.g., albumin, CRP, cytokines), and endpoint tissue samples for -omics analysis (e.g., transcriptomics).

An immunotoxicity use case can be modeled for testing inhaled OP exposure-response relationships. A broad spectrum of chronic conditions can be associated with occupational exposures. Some of these conditions are represented as increased incidence of common diseases like metabolic, cardiovascular, and neurologic disease. For many of these conditions, including the chronic neurodegenerative diseases, immune mechanisms have been postulated and several putative toxicants are possible. Noteworthy among these potential toxicant exposures are the OP chemicals that are ubiquitous as both pesticides and warfare agents. Epidemiology studies in agricultural pesticide applicators provide evidence that immunologic liabilities are possible with chronic exposures to OP chemicals. OP exposure is an example use case.

Immunotoxicity from exposures to drugs or chemicals can be acute or chronic, suppressive or stimulatory, direct or indirect, and involve any one of the tissue compartments of the immune system. Sentinel effector organs like the spleen are a point of biological convergence in the complex immune system providing an opportunity to reduce the natural complexity of this organ system to one that is tractable for modeling. A human-derived, multi-organ, in vitro, moderate-throughput system that models a common route of exposure (e.g., lung), metabolic activation or deactivation (e.g., liver) and immunologic responses (e.g., spleen) would provide a useful capability for detecting and characterizing potential immunologic liabilities of military or drug exposures. This system could also support the development of countermeasures.

The modeling system could be positioned as a primary screen for suspected immune toxicant agents where positive outcomes might prompt the addition of higher resolution immune endpoints in subsequent in vivo general toxicity studies. It could be a more human-relevant confirmation of signals detected in an initial general toxicity animal study or a more mechanistically informative investigative tool to better characterize those signals. This could also be a primary modeling system supporting the design of useful countermeasures with the potential to be more sensitive, specific, and human-relevant than traditional animal-based approaches.

Determining the acute physiological and adaptive immune responses to EVLP and TSST-1 Exposure (2-2.5 years at JHU). The interacting multiorgan MPS system will be challenged with either EVLP via the oral route e.g., intestinal MPS or the Staphylococcus aureus-derived TSST-1 toxin systemic exposure where the toxin is added to the universal media. Immune activation will be real-time monitored via glucose to lactate conversion and characterized more thoroughly every two days after partial media change. We will measure soluble factors identified as performance criteria for each tissue, and additionally survey a broad array of multiplexed cytokines indicative of the activation of antigen-presenting cells and T cell effector function/polarization such as IFN-g, TNF-a, Granzyme B, IL-1, IL-6, IL-12, IL-13, IL-17, IL-23. Additionally, the spleen compartment will be monitored via CFSE labeling indicative of T cell expansion. At the end of the experiment, cells will be harvested for bulk RNA sequencing and flow cytometry to determine CD4 and CD8 T cell responses. Media will be assessed based on the aforementioned metrics.

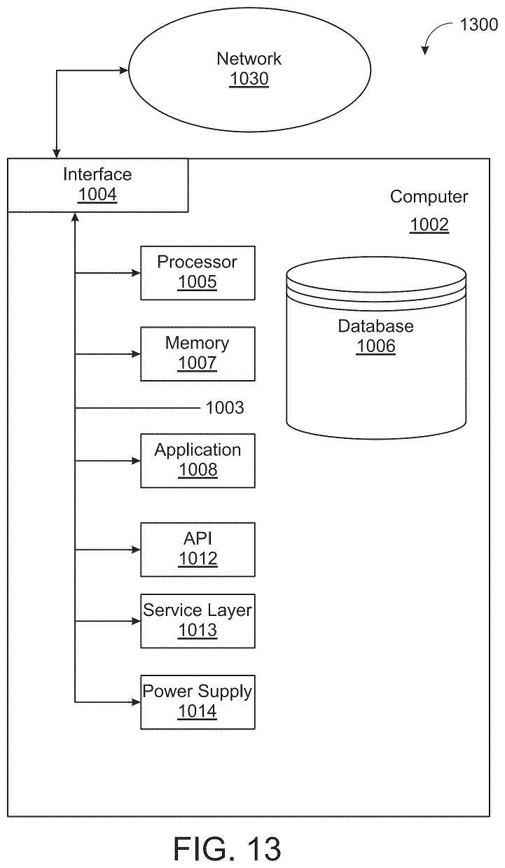

FIG. 13 is a block diagram of an example computer system 1300 used to provide computational functionalities associated with described algorithms, methods, functions, processes, flows, and procedures described in the present disclosure according to some implementations of the present disclosure. The illustrated computer 1002 is intended to encompass any computing device such as a server, a desktop computer, a laptop/notebook computer, a wireless data port, a smart phone, a personal data assistant (PDA), a tablet computing device, or one or more processors within these devices, including physical instances, virtual instances, or both. The computer 1002 can include input devices such as keypads, keyboards, and touch screens that can accept user information. Also, the computer 1002 can include output devices that can convey information associated with the operation of the computer 1002. The information can include digital data, visual data, audio information, or a combination of information. The information can be presented in a graphical user interface (UI or GUI).

The computer 1002 can serve in a role as a client, a network component, a server, a database, a persistency, or components of a computer system for performing the subject matter described in the present disclosure. The illustrated computer 1002 is communicably coupled with a network 1030. In some implementations, one or more components of the computer 1002 can be configured to operate within different environments, including cloud-computing-based environments, local environments, global environments, and combinations of environments.

At a high level, the computer 1002 is an electronic computing device operable to receive, transmit, process, store, and manage data and information associated with the described subject matter. According to some implementations, the computer 1002 can also include, or be communicably coupled with, an application server, an email server, a web server, a caching server, a streaming data server, or a combination of servers.

The computer 1002 can receive requests over network 1030 from a client application (for example, executing on another computer 1002). The computer 1002 can respond to the received requests by processing the received requests using software applications. Requests can also be sent to the computer 1002 from internal users (for example, from a command console), external (or third) parties, automated applications, entities, individuals, systems, and computers.

Each of the components of the computer 1002 can communicate using a system bus 1003. In some implementations, any or all of the components of the computer 1002, including hardware or software components, can interface with each other or the interface 1004 (or a combination of both), over the system bus 1003. Interfaces can use an application programming interface (API) 1012, a service layer 1013, or a combination of the API 1012 and service layer 1013. The API 1012 can include specifications for routines, data structures, and object classes. The API 1012 can be either computer-language independent or dependent. The API 1012 can refer to a complete interface, a single function, or a set of APIs.

The service layer 1013 can provide software services to the computer 1002 and other components (whether illustrated or not) that are communicably coupled to the computer 1002. The functionality of the computer 1002 can be accessible for all service consumers using this service layer. Software services, such as those provided by the service layer 1013, can provide reusable, defined functionalities through a defined interface. For example, the interface can be software written in JAVA, C++, or a language providing data in extensible markup language (XML) format. While illustrated as an integrated component of the computer 1002, in alternative implementations, the API 1012 or the service layer 1013 can be stand-alone components in relation to other components of the computer 1002 and other components communicably coupled to the computer 1002. Moreover, any or all parts of the API 1012 or the service layer 1013 can be implemented as child or sub-modules of another software module, enterprise application, or hardware module without departing from the scope of the present disclosure.

The computer 1002 includes an interface 1004. Although illustrated as a single interface 1004 in FIG. 13, two or more interfaces 1004 can be used according to particular needs, desires, or particular implementations of the computer 1002 and the described functionality. The interface 1004 can be used by the computer 1002 for communicating with other systems that are connected to the network 1030 (whether illustrated or not) in a distributed environment. Generally, the interface 1004 can include, or be implemented using, logic encoded in software or hardware (or a combination of software and hardware) operable to communicate with the network 1030. More specifically, the interface 1004 can include software supporting one or more communication protocols associated with communications. As such, the network 1030 or the interface's hardware can be operable to communicate physical signals within and outside of the illustrated computer 1002.

The computer 1002 includes a processor 1005. Although illustrated as a single processor 1005 in FIG. 13, two or more processors 1005 can be used according to particular needs, desires, or particular implementations of the computer 1002 and the described functionality. Generally, the processor 1005 can execute instructions and can manipulate data to perform the operations of the computer 1002, including operations using algorithms, methods, functions, processes, flows, and procedures as described in the present disclosure.

The computer 1002 also includes a database 1006 that can hold data for the computer 1002 and other components connected to the network 1030 (whether illustrated or not). For example, database 1006 can be an in-memory, conventional, or a database storing data consistent with the present disclosure. In some implementations, database 1006 can be a combination of two or more different database types (for example, hybrid in-memory and conventional databases) according to particular needs, desires, or particular implementations of the computer 1002 and the described functionality. Although illustrated as a single database 1006 in FIG. 6, two or more databases (of the same, different, or combination of types) can be used according to particular needs, desires, or particular implementations of the computer 1002 and the described functionality. While database 1006 is illustrated as an internal component of the computer 1002, in alternative implementations, database 1006 can be external to the computer 1002.

The computer 1002 also includes a memory 1007 that can hold data for the computer 1002 or a combination of components connected to the network 1030 (whether illustrated or not). Memory 1007 can store any data consistent with the present disclosure. In some implementations, memory 1007 can be a combination of two or more different types of memory (for example, a combination of semiconductor and magnetic storage) according to particular needs, desires, or particular implementations of the computer 1002 and the described functionality. Although illustrated as a single memory 1007 in FIG. 13, two or more memories 1007 (of the same, different, or combination of types) can be used according to particular needs, desires, or particular implementations of the computer 1002 and the described functionality. While memory 1007 is illustrated as an internal component of the computer 1002, in alternative implementations, memory 1007 can be external to the computer 1002.

The application 1008 can be an algorithmic software engine providing functionality according to particular needs, desires, or particular implementations of the computer 1002 and the described functionality. For example, application 1008 can serve as one or more components, modules, or applications. Further, although illustrated as a single application 1008, the application 1008 can be implemented as multiple applications 1008 on the computer 1002. In addition, although illustrated as internal to the computer 1002, in alternative implementations, the application 1008 can be external to the computer 1002.

The computer 1002 can also include a power supply 1014. The power supply 1014 can include a rechargeable or non-rechargeable battery that can be configured to be either user-or non-user-replaceable. In some implementations, the power supply 1014 can include power-conversion and management circuits, including recharging, standby, and power management functionalities. In some implementations, the power-supply 1014 can include a power plug to allow the computer 1002 to be plugged into a wall socket or a power source to, for example, power the computer 1002 or recharge a rechargeable battery.

There can be any number of computers 1002 associated with, or external to, a computer system containing computer 1002, with each computer 1002 communicating over network 1030. Further, the terms “client,” “user,” and other appropriate terminology can be used interchangeably, as appropriate, without departing from the scope of the present disclosure. Moreover, the present disclosure contemplates that many users can use one computer 1002 and one user can use multiple computers 1002.

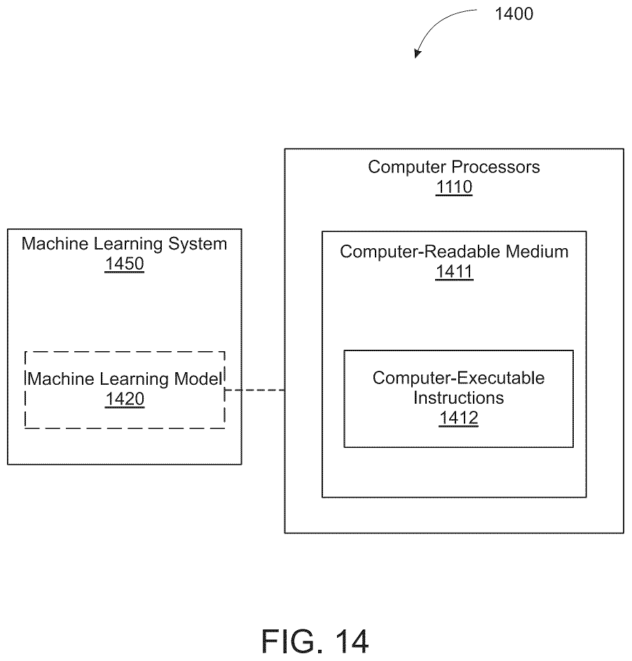

FIG. 14 is a diagram illustrating an example computer system 1400 configured to execute a machine learning model. Generally, the computer system 1400 is configured to process data indicating a phenotype and determine a class label (“healthy” or “disease”) to predict the effect of a target on the state of the MPS. The system 1400 includes computer processors 1410. The computer processors 1410 include computer-readable memory 1411 and computer readable instructions 1412. The system 1400 also includes a machine learning system 1450. The machine learning system 1450 includes a machine learning model 1420. The machine learning model 1420 can be separate from or integrated with the computer processors 1410.

The computer-readable medium 1411 (or computer-readable memory) can include any data storage technology type which is suitable to the local technical environment, including but not limited to semiconductor based memory devices, magnetic memory devices and systems, optical memory devices and systems, fixed memory, removable memory, disc memory, flash memory, dynamic random-access memory (DRAM), static random-access memory (SRAM), electronically erasable programmable read-only memory (EEPROM) and the like. In an embodiment, the computer-readable medium 1411 includes code-segment having executable instructions.

In some implementations, the computer processors 1410 include a general purpose processor. In some implementations, the computer processors 1410 include a central processing unit (CPU). In some implementations, the computer processors 1410 include at least one application specific integrated circuit (ASIC). The computer processors 1410 can also include general purpose programmable microprocessors, graphic processing units, special-purpose programmable microprocessors, digital signal processors (DSPs), programmable logic arrays (PLAs), field programmable gate arrays (FPGA), special purpose electronic circuits, etc., or a combination thereof. The computer processors 1410 are configured to execute program code means such as the computer-executable instructions 1412 and configured to execute executable logic that includes the machine learning model 1420.

The computer processors 1410 are configured to receive data indicating a molecular structure of, for example, a drug. The data can be obtained through one or more means, such as wireless communications with databases, optical fiber communications, USB, CD-ROM, and so forth.

The machine learning model 1420 is capable of processing the data to determine the class of phenotype (“healthy or disease”). In some implementations, the machine learning model 1420 is trained to determine class using a data set that includes MPS data (e.g., phenotypic, transcriptomic, etc.) and MPS labels. The machine learning model 1420 can classify the phenotype predicted by in vitro or in silico target perturbations. Accordingly, when a data set (in vitro or in silico) is introduced to the machine learning model 1420, it can predict whether an MPS platform exhibits a healthy or disease phenotype.

The machine learning system 1450 is capable of applying machine learning techniques to train the machine learning model 1420. As part of the training of the machine learning model 1420, the machine learning system 1450 forms a training set of input data by identifying a positive training set of input data items that have been determined to have the property in question, and, in some embodiments, forms a negative training set of input data items that lack the property in question.

The machine learning system 1450 extracts feature values from the input data of the training set, the features being variables deemed potentially relevant to whether or not the input data items have the associated property or properties. An ordered list of the features for the input data is herein referred to as the feature vector for the input data. In one embodiment, the machine learning system 1450 applies dimensionality reduction (e.g., via linear discriminant analysis (LDA), principle component analysis (PCA), or the like) to reduce the amount of data in the feature vectors for the input data to a smaller, more representative set of data.

In some implementations, the machine learning system 1450 uses supervised machine learning to train the machine learning models 1420 with the feature vectors of the positive training set and the negative training set serving as the inputs. Different machine learning techniques—such as linear support vector machine (linear SVM), boosting for other algorithms (e.g., AdaBoost), neural networks, logistic regression, naïve Bayes, memory-based learning, random forests, bagged trees, decision trees, boosted trees, or boosted stumps—may be used in different embodiments. The machine learning model 1420, when applied to the feature vector extracted from the input data item, outputs an indication of whether the input data item has the property in question, such as a Boolean yes/no estimate, or a scalar value representing a probability.

In some embodiments, a validation set is formed of additional input data, other than those in the training sets, which have already been determined to have or to lack the property in question. The machine learning system 1450 applies the trained machine learning model 1420 to the data of the validation set to quantify the accuracy of the machine learning model 1420. Common metrics applied in accuracy measurement include: Precision=TP/(TP+FP) and Recall=TP/(TP+FN), where precision is how many the machine learning model correctly predicted (TP or true positives) out of the total it predicted (TP+FP or false positives), and recall is how many the machine learning model correctly predicted (TP) out of the total number of input data items that did have the property in question (TP +FN or false negatives). The F score (F-score=2*PR/(P+R)) unifies precision and recall into a single measure. In one embodiment, the machine learning module iteratively re-trains the machine learning model until the occurrence of a stopping condition, such as the accuracy measurement indication that the model is sufficiently accurate, or a number of training rounds having taken place.

In some implementations, the machine learning model 1420 is a convolutional neural network (CNN). A CNN can be configured based on a presumption that inputs to the CNN correspond to image pixel data for an image or other data that includes features at multiple spatial locations. For example, sets of inputs can form a multi-dimensional data structure, such as a tensor, that represent color features of an example digital image (e.g., a biological image of biological tissue). In some implementations, inputs to the CNN correspond to a variety of other types of data, such as data obtained from different devices and sensors of a vehicle, point cloud data, audio data that includes certain features or raw audio at each of multiple time steps, or various types of one-dimensional or multiple dimensional data. A convolutional layer of the CNN can process the inputs to transform features of the image that are represented by inputs of the data structure. For example, the inputs are processed by performing dot product operations using input data along a given dimension of the data structure and a set of parameters for the convolutional layer.

Performing computations for a convolutional layer can include applying one or more sets of kernels to portions of inputs in the data structure. The manner in which CNN performs the computations can be based on specific properties for each layer of an example multi-layer neural network or deep neural network that supports deep neural net workloads. A deep neural network can include one or more convolutional towers (or layers) along with other computational layers. In particular, for example computer vision applications, these convolutional towers often account for a large proportion of the inference calculations that are performed. Convolutional layers of a CNN can have sets of artificial neurons that are arranged in three dimensions, a width dimension, a height dimension, and a depth dimension. The depth dimension corresponds to a third dimension of an input or activation volume and can represent respective color channels of an image. For example, input images can form an input volume of data (e.g., activations), and the volume has dimensions 32×32×3 (width, height, depth respectively). A depth dimension of 3 can correspond to the RGB color channels of red (R), green (G), and blue (B).

In general, layers of a CNN are configured to transform the three dimensional input volume (inputs) to a multi-dimensional output volume of neuron activations (activations). For example, a 3D input structure of 32×32×3 holds the raw pixel values of an example image, in this case an image of width 32, height 32, and with three color channels, R,G,B. A convolutional layer of a CNN of the machine learning model 1420 computes the output of neurons that may be connected to local regions in the input volume. Each neuron in the convolutional layer can be connected only to a local region in the input volume spatially, but to the full depth (e.g., all color channels) of the input volume. For a set of neurons at the convolutional layer, the layer computes a dot product between the parameters (weights) for the neurons and a certain region in the input volume to which the neurons are connected. This computation may result in a volume such as 32×32×12, where 12 corresponds to a number of kernels that are used for the computation. A neuron's connection to inputs of a region can have a spatial extent along the depth axis that is equal to the depth of the input volume. The spatial extent corresponds to spatial dimensions (e.g., x and y dimensions) of a kernel.

A set of kernels can have spatial characteristics that include a width and a height and that extends through a depth of the input volume. Each set of kernels for the layer is applied to one or more sets of inputs provided to the layer. That is, for each kernel or set of kernels, the machine learning model 1420 can overlay the kernel, which can be represented multi-dimensionally, over a first portion of layer inputs (e.g., that form an input volume or input tensor), which can be represented multi-dimensionally. For example, a set of kernels for a first layer of a CNN may have size 5×5×3×16, corresponding to a width of 5 pixels, a height of 5 pixel, a depth of 3 that corresponds to the color channels of the input volume to which to a kernel is being applied, and an output dimension of 16 that corresponds to a number of output channels. In this context, the set of kernels includes 16 kernels so that an output of the convolution has a depth dimension of 16.

The machine learning model 1420 can then compute a dot product from the overlapped elements. For example, the machine learning model 1420 can convolve (or slide) each kernel across the width and height of the input volume and compute dot products between the entries of the kernel and inputs for a position or region of the image. Each output value in a convolution output is the result of a dot product between a kernel and some set of inputs from an example input tensor. The dot product can result in a convolution output that corresponds to a single layer input, e.g., an activation element that has an upper-left position in the overlapped multi-dimensional space. As discussed above, a neuron of a convolutional layer can be connected to a region of the input volume that includes multiple inputs. The machine learning model 1420 can convolve each kernel over each input of an input volume. The machine learning model 1420 can perform this convolution operation by, for example, moving (or sliding) each kernel over each input in the region.

The machine learning model 1420 can move each kernel over inputs of the region based on a stride value for a given convolutional layer. For example, when the stride is set to 1, then the machine learning model 1420 can move the kernels over the region one pixel (or input) at a time. Likewise, when the stride is 2, then the machine learning model 1420 can move the kernels over the region two pixels at a time. Thus, kernels may be shifted based on a stride value for a layer and the machine learning model 1420 can repeatedly perform this process until inputs for the region have a corresponding dot product. Related to the stride value is a skip value. The skip value can identify one or more sets of inputs (2×2), in a region of the input volume, that are skipped when inputs are loaded for processing at a neural network layer. In some implementations, an input volume of pixels for an image can be “padded” with zeros, e.g., around a border region of an image. This zero-padding is used to control the spatial size of the output volumes.

As discussed previously, a convolutional layer of CNN is configured to transform a three dimensional input volume (inputs of the region) to a multi-dimensional output volume of neuron activations. For example, as the kernel is convolved over the width and height of the input volume, the machine learning model 1420 can produce a multi-dimensional activation map that includes results of convolving the kernel at one or more spatial positions based on the stride value. In some cases, increasing the stride value produces smaller output volumes of activations spatially. In some implementations, an activation can be applied to outputs of the convolution before the outputs are sent to a subsequent layer of the CNN.

An example convolutional layer can have one or more control parameters for the layer that represent properties of the layer. For example, the control parameters can include a number of kernels, K, the spatial extent of the kernels, F, the stride (or skip), S, and the amount of zero padding, P. Numerical values for these parameters, the inputs to the layer, and the parameter values of the kernel for the layer shape the computations that occur at the layer and the size of the output volume for the layer. In some implementations, the spatial size of the output volume is computed as a function of the input volume size, W, using the formula (W−F+2P)/S+1. For example, an input tensor can represent a pixel input volume of size [227×227×3]. A convolutional layer of a CNN can have a spatial extent value of F=11, a stride value of S=4, and no zero-padding (P=0). Using the above formula and a layer kernel quantity of K=116, the machine learning model 1420 performs computations for the layer that results in a convolutional layer output volume of size [55×55×156], where 55 is obtained from [(227−11+0)/4+1=55].

The computations (e.g., dot product computations) for a convolutional layer, or other layers, of a CNN involve performing mathematical operations, e.g., multiplication and addition, using a computation unit of a hardware circuit of the machine learning model 1420. The design of a hardware circuit can cause a system to be limited in its ability to fully utilize computing cells of the circuit when performing computations for layers of a neural network.

In the previous description, for the purposes of explanation, numerous specific details are set forth in order to provide a thorough understanding of the present disclosure. However, the present disclosure may be practiced without these specific details. In other instances, well-known structures and devices are shown in block diagram form in order to avoid unnecessarily obscuring the present disclosure.

In the drawings, specific arrangements or orderings of schematic elements, such as those representing devices, modules, instruction blocks and data elements, are shown for case of description. However, it should be understood by those skilled in the art that the specific ordering or arrangement of the schematic elements in the drawings is not meant to imply that a particular order or sequence of processing, or separation of processes, is required. Further, the inclusion of a schematic element in a drawing is not meant to imply that such element is required in all embodiments or that the features represented by such element may not be included in or combined with other elements in some implementations.

Further, in the drawings, where connecting elements, such as solid or dashed lines or arrows, are used to illustrate a connection, relationship, or association between or among two or more other schematic elements, the absence of any such connecting elements is not meant to imply that no connection, relationship, or association can exist. In other words, some connections, relationships, or associations between elements are not shown in the drawings so as not to obscure the disclosure. In addition, for case of illustration, a single connecting element is used to represent multiple connections, relationships or associations between elements. For example, where a connecting element represents a communication of signals, data, or instructions, it should be understood by those skilled in the art that such clement represents one or multiple signal paths (e.g., a bus), as may be needed, to affect the communication.

Reference is made in detail to implementations, examples of which are illustrated in the accompanying drawings. In the previous description, numerous specific details are set forth in order to provide a thorough understanding of the various described implementations. However, it are apparent to one of ordinary skill in the art that the various described implementations may be practiced without these specific details. In other instances, well-known methods, procedures, components, circuits, and networks have not been described in detail so as not to unnecessarily obscure aspects of the implementations.

Several features are described that can each be used independently of one another or with any combination of other features. However, any individual feature may not address any of the problems discussed above or might only address one of the problems discussed above. Some of the problems discussed above might not be fully addressed by any of the features described in this specification. Although headings are provided, data related to a particular heading, but not found in the section having that heading, may also be found elsewhere in this description.

Implementations of the subject matter and the functional operations described in this specification can be implemented in digital electronic circuitry, in tangibly embodied computer software or firmware, in computer hardware, including the structures disclosed in this specification and their structural equivalents, or in combinations of one or more of them. Software implementations of the described subject matter can be implemented as one or more computer programs. Each computer program can include one or more modules of computer program instructions encoded on a tangible, non-transitory, computer-readable computer-storage medium for execution by, or to control the operation of, data processing apparatus. Alternatively, or additionally, the program instructions can be encoded in/on an artificially generated propagated signal. For example, the signal can be a machine-generated electrical, optical, or electromagnetic signal that is generated to encode information for transmission to suitable receiver apparatus for execution by a data processing apparatus. The computer-storage medium can be a machine-readable storage device, a machine-readable storage substrate, a random or serial access memory device, or a combination of computer-storage mediums.

The terms “data processing apparatus,” “computer,” and “electronic computer device” (or equivalent as understood by one of ordinary skill in the art) refer to data processing hardware. For example, a data processing apparatus can encompass all kinds of apparatus, devices, and machines for processing data, including by way of example, a programmable processor, a computer, or multiple processors or computers. The apparatus can also include special purpose logic circuitry including, for example, a central processing unit (CPU), a field programmable gate array (FPGA), or an application specific integrated circuit (ASIC). In some implementations, the data processing apparatus or special purpose logic circuitry (or a combination of the data processing apparatus or special purpose logic circuitry) can be hardware- or software-based (or a combination of both hardware- and software-based). The apparatus can optionally include code that creates an execution environment for computer programs, for example, code that constitutes processor firmware, a protocol stack, a database management system, an operating system, or a combination of execution environments. The present disclosure contemplates the use of data processing apparatuses with or without conventional operating systems, for example, LINUX, UNIX, WINDOWS, MAC OS, ANDROID, or IOS.

A computer program, which can also be referred to or described as a program, software, a software application, a module, a software module, a script, or code, can be written in any form of programming language. Programming languages can include, for example, compiled languages, interpreted languages, declarative languages, or procedural languages. Programs can be deployed in any form, including as stand-alone programs, modules, components, subroutines, or units for use in a computing environment. A computer program can, but need not, correspond to a file in a file system. A program can be stored in a portion of a file that holds other programs or data, for example, one or more scripts stored in a markup language document, in a single file dedicated to the program in question, or in multiple coordinated files storing one or more modules, sub programs, or portions of code. A computer program can be deployed for execution on one computer or on multiple computers that are located, for example, at one site or distributed across multiple sites that are interconnected by a communication network. While portions of the programs illustrated in the various figures may be shown as individual modules that implement the various features and functionality through various objects, methods, or processes, the programs can instead include a number of sub-modules, third-party services, components, and libraries. Conversely, the features and functionality of various components can be combined into single components as appropriate. Thresholds used to make computational determinations can be statically, dynamically, or both statically and dynamically determined.

The methods, processes, or logic flows described in this specification can be performed by one or more programmable computers executing one or more computer programs to perform functions by operating on input data and generating output. The methods, processes, or logic flows can also be performed by, and apparatus can also be implemented as, special purpose logic circuitry, for example, a CPU, an FPGA, or an ASIC.

Computers suitable for the execution of a computer program can be based on one or more of general and special purpose microprocessors and other kinds of CPUs. The elements of a computer are a CPU for performing or executing instructions and one or more memory devices for storing instructions and data. Generally, a CPU can receive instructions and data from (and write data to) a memory. A computer can also include, or be operatively coupled to, one or more mass storage devices for storing data. In some implementations, a computer can receive data from, and transfer data to, the mass storage devices including, for example, magnetic, magneto optical disks, or optical disks. Moreover, a computer can be embedded in another device, for example, a mobile telephone, a personal digital assistant (PDA), a mobile audio or video player, a game console, a global positioning system (GPS) receiver, or a portable storage device such as a universal serial bus (USB) flash drive.

Computer readable media (transitory or non-transitory, as appropriate) suitable for storing computer program instructions and data can include all forms of permanent/non- permanent and volatile/non-volatile memory, media, and memory devices. Computer readable media can include, for example, semiconductor memory devices such as random access memory (RAM), read only memory (ROM), phase change memory (PRAM), static random access memory (SRAM), dynamic random access memory (DRAM), erasable programmable read-only memory (EPROM), electrically erasable programmable read-only memory (EEPROM), and flash memory devices. Computer readable media can also include, for example, magnetic devices such as tape, cartridges, cassettes, and internal/removable disks. Computer readable media can also include magneto optical disks and optical memory devices and technologies including, for example, digital video disc (DVD), CD ROM, DVD+/−R, DVD-RAM, DVD-ROM, HD-DVD, and BLURAY. The memory can store various objects or data, including caches, classes, frameworks, applications, modules, backup data, jobs, web pages, web page templates, data structures, database tables, repositories, and dynamic information. Types of objects and data stored in memory can include parameters, variables, algorithms, instructions, rules, constraints, and references. Additionally, the memory can include logs, policies, security or access data, and reporting files. The processor and the memory can be supplemented by, or incorporated in, special purpose logic circuitry.

Implementations of the subject matter described in the present disclosure can be implemented on a computer having a display device for providing interaction with a user, including displaying information to (and receiving input from) the user. Types of display devices can include, for example, a cathode ray tube (CRT), a liquid crystal display (LCD), a light-emitting diode (LED), and a plasma monitor. Display devices can include a keyboard and pointing devices including, for example, a mouse, a trackball, or a trackpad. User input can also be provided to the computer through the use of a touchscreen, such as a tablet computer surface with pressure sensitivity or a multi-touch screen using capacitive or electric sensing. Other kinds of devices can be used to provide for interaction with a user, including to receive user feedback including, for example, sensory feedback including visual feedback, auditory feedback, or tactile feedback. Input from the user can be received in the form of acoustic, speech, or tactile input. In addition, a computer can interact with a user by sending documents to, and receiving documents from, a device that is used by the user. For example, the computer can send web pages to a web browser on a user's client device in response to requests received from the web browser.

The term “graphical user interface,” or “GUI,” can be used in the singular or the plural to describe one or more graphical user interfaces and each of the displays of a particular graphical user interface. Therefore, a GUI can represent any graphical user interface, including, but not limited to, a web browser, a touch screen, or a command line interface (CLI) that processes information and efficiently presents the information results to the user. In general, a GUI can include a plurality of user interface (UI) elements, some or all associated with a web browser, such as interactive fields, pull-down lists, and buttons. These and other UI elements can be related to or represent the functions of the web browser.

Implementations of the subject matter described in this specification can be implemented in a computing system that includes a back end component (for example, as a data server), or that includes a middleware component (for example, an application server). Moreover, the computing system can include a front-end component, for example, a client computer having one or both of a graphical user interface or a Web browser through which a user can interact with the computer. The components of the system can be interconnected by any form or medium of wireline or wireless digital data communication (or a combination of data communication) in a communication network. Examples of communication networks include a local area network (LAN), a radio access network (RAN), a metropolitan area network (MAN), a wide area network (WAN), Worldwide Interoperability for Microwave Access (WIMAX), a wireless local area network (WLAN) (for example, using 802.11 a/b/g/n or 802.20 or a combination of protocols), all or a portion of the Internet, or any other communication system or systems at one or more locations (or a combination of communication networks). The network can communicate with, for example, Internet Protocol (IP) packets, frame relay frames, asynchronous transfer mode (ATM) cells, voice, video, data, or a combination of communication types between network addresses.

The computing system can include clients and servers. A client and server can generally be remote from each other and can typically interact through a communication network. The relationship of client and server can arise by virtue of computer programs running on the respective computers and having a client-server relationship.

Cluster file systems can be any file system type accessible from multiple servers for read and update. Locking or consistency tracking may not be necessary since the locking of exchange file system can be done at application layer. Furthermore, Unicode data files can be different from non-Unicode data files.

While this specification contains many specific implementation details, these should not be construed as limitations on the scope of what may be claimed, but rather as descriptions of features that may be specific to particular implementations. Certain features that are described in this specification in the context of separate implementations can also be implemented, in combination, in a single implementation. Conversely, various features that are described in the context of a single implementation can also be implemented in multiple implementations, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

In the foregoing description, embodiments of the invention have been described with reference to numerous specific details that may vary from implementation to implementation. The description and drawings are, accordingly, to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction. Any definitions expressly set forth herein for terms contained in such claims shall govern the meaning of such terms as used in the claims. In addition, when we use the term “further comprising” or “further including” in the foregoing description or following claims, what follows this phrase can be an additional step or entity, or a sub-step/sub-entity of a previously-recited step or entity.

Particular implementations of the subject matter have been described. Other implementations, alterations, and permutations of the described implementations are within the scope of the following claims as are apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results. In certain circumstances, multitasking or parallel processing (or a combination of multitasking and parallel processing) may be advantageous and performed as deemed appropriate.

Moreover, the separation or integration of various system modules and components in the previously described implementations should not be understood as requiring such separation or integration in all implementations, and it should be understood that the described program components and systems can generally be integrated together in a single software product or packaged into multiple software products.

Accordingly, the previously described example implementations do not define or constrain the present disclosure. Other changes, substitutions, and alterations are also possible without departing from the spirit and scope of the present disclosure.

Furthermore, any claimed implementation is considered to be applicable to at least a computer-implemented method; a non-transitory, computer-readable medium storing computer-readable instructions to perform the computer-implemented method; and a computer system comprising a computer memory interoperably coupled with a hardware processor configured to perform the computer-implemented method or the instructions stored on the non-transitory, computer-readable medium.

A number of embodiments of these systems and methods have been described. Nevertheless, it are understood that various modifications may be made without departing from the scope of this disclosure.

What is claimed is:

1. A multiple tissue chamber (MTC) system, comprising:
a first tissue chamber comprising at least a first tissue;
a second tissue chamber comprising at least a second tissue different from the first tissue;
an oxygenation chamber for oxygenation of fluid media configured to flow in a fluid loop through the first tissue chamber and the second tissue chamber,
wherein the fluid media comprises immune cells;
a pump configured for causing recirculation of the fluid media including the immune cells through the first tissue chamber and the second tissue chamber; and
one or more sensors configured to measure an effect of the immune cells at the first tissue, the second tissue, or both the first tissue and the second tissue.

2. The system of claim 1, further comprising one or more sensors for trans-epithelial electrical resistance (TEER) monitoring.

3. The system of claim 1, further comprising one or more oxygen sensors configured to detect liver-specific adverse effects of mitochondrial toxicity.

4. The system of claim 1, further comprising glucose or lactate sensors configured to provide data for performance of immune assessments including T-cell activation for evaluation of adverse outcomes of CBRN threats.

5. The system of claim 1, wherein the first tissue comprises human bronchial epithelial cells (NHBEC) on top of hydrogel-embedded human lung fibroblasts.

6. The system of claim 1, wherein the second tissue comprises CD45+ tissue-resident immune cells from spleen red and white pulp.

7. The system of claim 1, further comprising a third tissue chamber comprising a third tissue, the third tissue comprising primary small-intestinal epithelial cells expanded as organoids and seeded as monolayers with CD45+ tissue-resident immune cells from an intestinal lamina propria.

8. The system of claim 1, wherein the first tissue chamber or the second tissue chamber or both comprise a milli-fluidic format enabling a repeated media sampling from basal recirculation and apical compartments to evaluate exposure-response relationships and- omics analysis to investigate secreted biomarkers.

9. The system of claim 1, wherein the first tissue comprises lung tissue, the second tissue comprises liver tissue, and wherein a third tissue chamber comprises spleen tissue.

10. The system of claim 1, wherein the first tissue comprises gut tissue, the second tissue comprises liver tissue, and wherein a third tissue chamber comprises spleen tissue.

11. The system of claim 1, further comprising a controller for receiving data from the one or more sensors to determine an immune response from the first tissue or the second tissue.

12. The system of claim 11, wherein the controller is configured to perform quantification of on-chip pharmacokinetic (PK) parameters including hepatic metabolism, uptake & disposition, tubular secretion & reabsorption, and muscle disposition.

13. The system of claim 12, wherein the controller is further configured to scale the quantified on-chip pharmacokinetic (PK) parameters.

14. A multiple tissue chamber (MTC) system, comprising:
a first modular micro-physiological system (MPS) system including a tissue chamber comprising at least a first tissue, the first MPS being removable from the MTC;
a second MPS including a second tissue chamber comprising at least a second tissue, the second MPS being removable from the MTC, the second tissue being different from the first tissue;
an oxygenation chamber for oxygenation of fluid media configured to recirculate in a fluid loop through the first tissue chamber and the second tissue chamber,
a pump configured for causing recirculation of the fluid media through the first tissue chamber and the second tissue chamber; and
one or more sensors configured to measure a parameter of the first tissue, the second tissue, or both the first tissue and the second tissue.

15. The MTC system of claim 14, further comprising:
a controller configured to host one or more instances of the MTC system and control recirculation of the fluid media or measurement by the one or more sensors of each MTC instance.

16. The MTC system of claim 14, wherein the one or more sensors are configured to send data to a client device in real-time or near real-time for analysis concurrent with the recirculation of the fluid media.

17. The MTC system of claim 14, wherein the one or more sensors comprise electrochemical aptamer-based (E-AB) sensors 322. Electrochemical aptamer-based (E-AB) biosensors for real-time biomarker monitoring.

18. The MTC system of claim 14, wherein the one or more sensors a sensor array for multianalyte monitoring in real-time.

19. The system of claim 14, wherein the first tissue comprises brain tissue, the second tissue comprises liver tissue, and wherein a third tissue chamber comprises lung tissue.

20. The system of claim 14, wherein the first tissue comprises gut tissue, the second tissue comprises liver tissue, and wherein a third tissue chamber comprises lung tissue.
